SEQUENCE LISTING (19 sequences)

SEQ ID NO: 1 - DNA, 3374 bp, Homo sapiens
  CDS (403)..(2256)
gccctttcttatgagcatgcctgtgttgggttgacagtgagggtaataatgacttgttggttgattgtagatatagggctctcccttgcaaggtaattaggctccttaaattacctgtaagattttcttgccacagcatccattctggttaggctggtgatcttctgagtagtgatagattggttggtggtgaggtttacaggtgttcccttctcttactcctggtgttggctacaatcaggtggcgtctagagcagcatgggacaggtgggtaaggggagtcttctcattatgcagaagtgatcaacttaaatctctgtcagatctacctttatgtagcccggcagtcgcgcggattgagcgggctcgcggcgctgggttcctggtctccgggccagggcaatgttccgcacggcagtgatgatggcggccagcctggcgctgaccggggctgtggtggctcacgcctactacctcaaacaccagttctaccccactgtggtgtacctgaccaagtccagccccagcatggcagtcctgtacatccaggcctttgtccttgtcttccttctgggcaaggtgatgggcaaggtgttctttgggcaactgagggcagcagagatggagcaccttctggaacgttcctggtacgccgtcacagagacttgtctggccttcaccgtttttcgggatgacttcagcccccgctttgttgcactcttcactcttcttctcttcctcaaatgtttccactggctggctgaggaccgtgtggactttatggaacgcagccccaacatctcctggctctttcactgccgcattgtctctcttatgttcctcctgggcatcctggacttcctcttcgtcagccacgcctatcacagcatcctgacccgtggggcctctgtgcagctggtgtttggctttgagtatgccatcctgatgacgatggtgctcaccatcttcatcaagtatgtgctgcactccgtggacctccagagtgagaacccctgggacaacaaggctgtgtacatgctctacacagagctgtttacaggcttcatcaaggttctgctgtacatggccttcatgaccatcatgatcaaggtgcacaccttcccactctttgccatccggcccatgtacctggccatgagacagttcaagaaagctgtgacagatgccatcatgtctcgccgagccatccgcaacatgaacaccctgtatccagatgccaccccagaggagctccaggcaatggacaatgtctgcatcatctgccgagaagagatggtgactggtgccaagagactgccctgcaaccacattttccataccagctgcctgcgctcctggttccagcggcagcagacctgccccacctgccgtatggatgtccttcgtgcatcgctgccagcgcagtcaccaccacccccggagcctgcggatcaggggccaccccctgccccccaccccccaccactcttgcctcagccccccaacttcccccagggcctcctgcctccttttcctccaggcatgttcccactgtggccccccatgggcccctttccacctgtcccgcctccccccagctcaggagaggctgtggctcctccatccaccagtgcagcagccctttctcggcccagtggagcagctacaaccacagctgctggcaccagtgctactgctgcttctgccacagcatctggcccaggctctggctctgccccagaggctggccctgcccctggtttccccttccctcctccctggatgggtatgcccctgcctccaccctttgccttccccccaatgcctgtgccccctgcgggctttgctgggctgaccccagaggagctacgagctctggagggccatgagcggcagcacctggaggcccggctgcagagcctgcgtaacatccacacactgctggacgccgccatgctgcagatcaaccagtacctcaccgtgctggcctccttggggcccccccggcctgccacttcagtcaactccactgaggggactgccactacagttgttgctgctgcctcctccaccagcatccctagctcagaggccacgaccccaaccccaggagcctccccaccagcccctgaaatggaaaggcctccagctcctgagtcagtgggcacagaggagatgcctgaggatggagagcccgatgcagcagagctccgccggcgccgcctgcagaagctggagtctcctgttgcccactgacactgccccagcccagccccagcctctgctcttttgagcagccctcgctggaacatgtcctgccaccaagtgccagctccctctctgtctgcaccagggagtagtacccccagctctgagaaagaggcggcatcccctaggccaagtggaaagaggctggggttcccatttgactccagtcccaggcagccatggggatctcgggtcagttccagccttcctctccaactcttcagccctgtgttctgctggggccatgaaggcagaaggtttagcctctgagaagccctcttcttcccccacccctttccaggagaaggggctgcccctccaagccctacttgtatgtgcggagtcacactgcagtgccgaacagtattagctcccgttcccaagtgtggactccagaggggctggaggcaagctatgaacttgctcgctggcccacccctaagactggtacccatttccttttcttaccctgatctccccagaagcctcttgtggtggtggctgtgccccctatgccctgtggcatttctgcgtcttactggcaaccacacaactcagggaaaggaatgcctgggagtgggggtgcaggcgggcagcactgagggaccctgccccgcccctccccccaggcccctttcccctgcagcttctcaagtgagactgacctgtctcacccagcagccactgcccagccgcactccaggcaagggccagtgcgcctgctcctgaccactgcaatcccagcgcccaaggaaggccacttctcaactggcagaacttctgaagtttagaattggaattacttccttactagtgtcttttggcttaaattttgtcttttgaagttgaatgcttaatcccgggaaagaggaacaggagtgccagactcctggtctttccagtttagaaaaggctctgtgccaaggagggaccacaggagctgggacctgcctgcccctgtcctttccccttggttttgtgttacaagagttgttggagacagtttcagatgattatttaatttgtaaatattgtacaaattttaatagcttaaattgtatatacagccaaataaaaacttgcattaacaaaaaaaaaaaaaaaaa

SEQ ID NO: 2 - protein, 617 aa, Homo sapiens [sequence omitted]

SEQ ID NO: 3 - DNA, 23 bp, Homo sapiens
aatgtctgcatcatctgccgaga

SEQ ID NO: 4 - DNA, 23 bp, Homo sapiens
aagctgtgacagatgccatcatg

SEQ ID NO: 5 - DNA, 23 bp, Homo sapiens
aaagctgtgacagatgccatcat

SEQ ID NO: 6 - DNA, 23 bp, Homo sapiens
aagaaagctgtgacagatgccat

SEQ ID NO: 7 - DNA, 23 bp, Homo sapiens
aaggttctgctgtacatggcctt

SEQ ID NO: 8 - DNA, 23 bp, Homo sapiens
aacaaggctgtgtacatgctcta

SEQ ID NO: 9 - DNA, 23 bp, Homo sapiens
aaatgtttccactggctggctga

SEQ ID NO: 10 - DNA, 23 bp, Homo sapiens
aaggtgttctttgggcaactgag

SEQ ID NO: 11 - DNA, 23 bp, Homo sapiens
aacatccacacactgctggacgc

SEQ ID NO: 12 - DNA, 23 bp, Homo sapiens
aacaccctgtatccagatgccac

SEQ ID NO: 13 - DNA, 23 bp, Homo sapiens
aaggtgcacaccttcccactctt

SEQ ID NO: 14 - DNA, 23 bp, Homo sapiens
aatgtttccactggctggctgag

SEQ ID NO: 15 - DNA, 23 bp, Homo sapiens
aagagactgccctgcaaccacat

SEQ ID NO: 16 - DNA, 23 bp, Homo sapiens
aacgttcctggtacgccgtcaca

SEQ ID NO: 17 - protein, 393 aa, Homo sapiens [sequence omitted]

SEQ ID NO: 18 - DNA, 21 bp, Artificial
  synthetic oligonucleotide sequence (DNA/RNA mixture)
gguguucuuugggcaacugtt

SEQ ID NO: 19 - DNA, 21 bp, Artificial
  sythetic oligonucleotide sequence (DNA/RNA mixture)
caguugcccaaagaacacctt

(19) United States
(12) Patent Application Publication
(10) Pub. No.: US 2008/0305102 A1
(43) Pub. Date: Dec. 11, 2008

(54) Therapeutic Agent for Cancer Comprising Substance Capable of Inhibiting Expression or Function of Synoviolin as Active Ingredient and Screening Method for the Therapeutic Agent for Cancer

(76) Inventors: Toshihiro Nakajima, Kanagawa (JP); Yukihiro Kato, Kanagawa (JP); Tadayuki Yamadera, Kanagawa (JP); Tetsuya Amano, Kanagawa (JP)

Correspondence Address: FOLEY & LARDNER LLP, 111 HUNTINGTON AVENUE, 26TH FLOOR, BOSTON, MA, 02199-7610, US

(21) Appl. No.: 11/993,774

(22) PCT Filed: Jun. 23, 2006
(86) PCT No.: PCT/JP2006/312582

§ 371 Date: Aug. 7, 2008

(30) Foreign Application Priority Data
Jun. 23, 2005 (JP) 2005-184092

(51) Int. Cl. A61K 39/395; C07H 21/00; C07K 16/18; C07K 2/00; C12Q 1/68; G01N 33/50; A61K 31/53; C07D 403/04; C07D 251/54; A61P 35/00; A61K 38/00; A61K 48/00

(52) U.S. Cl. 424/130.1; 536/24.5; 530/387.1; 530/300; 435/6; 435/4; 544/198; 544/197; 514/2; 514/44; 514/245

(57) ABSTRACT

Inhibition of synoviolin function was found to activate the cancer-suppressing protein p53. Substances inhibiting the function of synoviolin are useful as cancer therapeutic agents. The inhibition of synoviolin function was also found to lead to the inhibition of p53 ubiquitination, increased activity of p53 phosphorylation proteins, and the like. Based on these findings, the present invention provides methods capable of efficiently screening for cancer therapeutic agents. Further, it was also found that regulation of the autoubiquitination of synoviolin protein suppresses the proliferation of rheumatoid arthritis synovial cells. Substances regulating the autoubiquitination of synoviolin protein are useful as anti-rheumatic agents. Moreover, the present invention provides methods of efficiently screening for anti-rheumatic agents.

### TECHNICAL FIELD

The present invention relates to p53 protein activators that comprise substances that inhibit the expression or function of synoviolin protein as active ingredients, therapeutic agents for cancer that include p53 protein activators as active ingredients, and methods of screening for therapeutic agents for cancer.

Moreover, the present invention relates to anti-rheumatic agents comprising substances that regulate the autoubiquitination of synoviolin protein as active ingredients, and methods of screening for anti-rheumatic agents.

### BACKGROUND ART

Synoviolin is a novel protein that was discovered as a membrane protein and that is overexpressed in rheumatoid patient-derived synovial cells (see Patent Document 1). Studies using genetically modified animals have revealed that synoviolin is an essential molecule for the onset of rheumatoid arthritis.

A protein structure prediction system has suggested that synoviolin has a RING finger motif. This motif is often found in an enzyme, termed E3 ubiquitin ligase, which plays an important role in the ubiquitination of proteins. In fact, synoviolin has been proven to have an autoubiquitination activity, which is one of the characteristics of E3 ubiquitin ligase (see Patent Document 1).

The p53 gene is located on chromosome 17 at p 13 and is a cancer suppressor gene that is very important in the development and proliferation of cancer cells. The p53 protein recognizes a specific nucleotide sequence (5′-(A/T)GPyPyPy-3′) in DNA, and promotes the transcriptional activation of specific genes such as those encoding p21, GADD45, and BAX. The p53 protein is also known to have other physiological functions, including (i) suppressing the transcription of many other genes; (ii) binding to viral oncogene products such as SV40 large T antigen, adenovirus EIB protein, papilloma virus E6, and cellular oncogene products such as mdm2; and (iii) specifically binding to DNA having mismatches.

### [Patent Document 1] WO 02/052007

### DISCLOSURE OF THE INVENTION

### Problems to be Solved by the Invention

An objective of the present invention is to provide agents for suppressing cancer. More specifically, the objective is to provide p53 protein-activating agents, cancer therapeutic agents that comprise the activating agents as active ingredients, and methods of screening for cancer therapeutic agents. Another objective of the present invention is to provide anti-rheumatic agents comprising substances that regulate the autoubiquitination of synoviolin protein as active ingredients, and methods of screening for anti-rheumatic agents.

### Means for Solving the Problems

The present inventors conducted dedicated research to solve the above problems. As a result of detailed analysis on synoviolin homozygous knockout animals, a large number of cells undergoing apoptosis were observed compared with the wild-type animals, and it was revealed that the p53 protein, which is strongly associated with apoptosis induction, was localized in the nuclei and was strongly expressed therein. Moreover, it was discovered that inhibition of synoviolin function activated the p53 cancer suppressor gene or the p53 cancer suppressor protein to inhibit the proliferation of cancer cells.

The present inventors conducted research in more detail to elucidate the mechanism of p53 protein activation arising from the inhibition of synoviolin function.

The present inventors then discovered that inhibition of the expression or function of synoviolin protein leads to the phosphorylation of p53 by a kinase, thereby activating p53.

Moreover, it was discovered that inhibition of the expression or function of synoviolin led to inhibition of the ubiquitination of p53, resulting in the activation of p53 protein.

Furthermore, the present inventors showed that inhibition of the expression or function of synoviolin led to the inhibition of the binding between synoviolin and the p53 protein, resulting in activation of the p53 protein. Moreover, they succeeded in determining the p53 protein-binding site in synoviolin.

The above findings discovered by the present inventors demonstrate that substances that inhibit the expression or function of the synoviolin protein are useful as p53 protein activators, and that the activators can be therapeutic agents for cancer.

Furthermore, based on various findings discovered by the present application, the present inventors aimed to develop novel cancer therapeutic agents and made an attempt to obtain substances that inhibit the ubiquitination of p53 protein by synoviolin. As a result, they in fact succeeded in obtaining low molecular weight compounds termed compounds X and Y as substances that inhibit p53 protein ubiquitination.

Next, the influence of compounds X and Y on the cell proliferation of cultured cancer cells was examined. In these experiments, it was observed that the proliferation of cancer cells was suppressed, and compound Y increased the expression level of p53 protein in cancer cells. Compounds X and Y were suggested to increase the p53 protein expression level in cultured cancer cells by inhibiting the p53 ubiquitination reaction by synoviolin.

Specifically, compounds X and Y are considered to inhibit p53 ubiquitination by synoviolin and compounds X and Y thereby both increase p53 protein expression levels and exert an anticancer effect. These compounds are also expected to be clinically applied in combined modality therapies to enhance radiation sensitivity, in turn improving therapeutic efficiency by inducing the stabilization and intensive nuclear localization of p53.

The above findings show that these low molecular weight compounds are useful as cancer therapeutic agents, and that cancer therapeutic agents can be efficiently screened based on the findings discovered by the present inventors.

As described above, the present inventors succeeded in developing p53 protein activators comprising substances that inhibit the expression or function of the synoviolin protein, cancer therapeutic agents that comprise these activators, and screening methods for cancer therapeutic agents, thereby completing the present invention.

Specifically, the present invention relates to p53 protein activators, cancer therapeutic agents that comprise the activators as active ingredients, and screening methods for the cancer therapeutic agents. More specifically, the present invention provides:

[1] a p53 protein-activating agent, comprising as an active ingredient a substance that inhibits expression and/or function of a synoviolin protein;
[2] the agent of [1], wherein the substance that inhibits the expression and/or function is a low molecular weight compound having an activity of inhibiting the binding between a synoviolin protein and a p53 protein;
[3] the agent of [1], wherein the substance that inhibits the expression and/or function is a low molecular weight compound having a function of inhibiting ubiquitination of a p53 protein;
[4] the agent of [1], wherein the substance that inhibits the expression and/or function is a low molecular weight compound having a function of activating a p53 phosphorylation protein;
[5] the agent of [4], wherein the p53 phosphorylation protein is ATM or ATR;
[6] the agent of [1], wherein the substance that inhibits the expression and/or function is a compound selected from the group consisting of:

(a) an antisense nucleic acid against a transcript of a synoviolin gene or a portion of the transcript;

(b) a nucleic acid having a ribozyme activity of specifically cleaving a transcript of a synoviolin gene; and

(c) a nucleic acid having an effect of inhibiting the expression of a synoviolin gene by an RNAi effect;

[7] the agent of [1], wherein the substance that inhibits the expression and/or function is a compound of (a) or (b):

(a) an antibody which binds to a synoviolin protein and/or a p53 protein; and

(b) a synoviolin protein mutant having a dominant negative property against the synoviolin protein;

[8] a cancer therapeutic agent, comprising the agent for activating a p53 protein of any one of [1] to [7] as an active ingredient;
[9] a method of screening for a cancer therapeutic agent, comprising the steps of:

(a) contacting a test compound with a cell expressing a synoviolin gene;

(b) measuring expression level or activity of a synoviolin protein in the cell; and

(c) selecting a compound that lowers the expression level or activity as compared to when the test compound is not contacted;

[10] a method of screening for a cancer therapeutic agent, comprising the steps of:

(a) contacting a test compound with a cell or cell extract which contains DNA having a structure in which a transcriptional regulatory region of a synoviolin gene and a reporter gene are operably linked to each other;

(b) measuring the expression level of the reporter gene; and

(c) selecting a compound that lowers the expression level of the reporter gene as compared to when the test compound is not contacted;

[11] a method of screening for a cancer therapeutic agent, comprising the steps of;

(a) contacting a synoviolin protein, a p53 protein, and a test compound;

(b) measuring the binding activity between the synoviolin protein and the p53 protein; and

(c) selecting a compound that lowers the binding activity as compared to when the test compound is not contacted;

[12] a method of screening for a cancer therapeutic agent, comprising the steps of:

(a) contacting a synoviolin protein, a p53 protein, and a test compound;

(b) measuring ubiquitination of the p53 protein; and

(c) selecting a compound that lowers the ubiquitination as compared to when the test compound is not contacted;

[13] a method of screening for a cancer therapeutic agent, comprising the steps of:

(a) contacting a synoviolin protein, a p53 protein, a p53 phosphorylation protein, and a test compound;

(b) measuring phosphorylation activity of the p53 phosphorylation protein which uses the p53 protein as a substrate; and

(c) selecting a compound that increases the phosphorylation activity as compared to when the test compound is not contacted;

[14] the method of [13], wherein the p53 phosphorylation protein is ATM or ATR; and
[15] a kit for screening for a cancer therapeutic agent, comprising as components:

(a) a synoviolin protein; and

(b) a p53 protein.

Furthermore, the present invention provides methods for preventing or treating cancer, comprising the step of administering a p53 protein activator of the present invention or a cancer therapeutic agent comprising a p53 protein activator as an active ingredient, and the use of the p53 protein activators of the present invention in the production of cancer therapeutic agents.

Moreover, the present inventors constructed a system for evaluating the autoubiquitination reaction of the synoviolin protein by means of ELISA, thereby discovering two low molecular weight compounds, No. 32 (compound X) and No. 38 (compound Y), that suppressed the proliferation of rheumatoid arthritis synovial cells (RASCs) at low concentrations.

The above findings show that these low molecular weight compounds are useful as anti-rheumatic agents and that anti-rheumatic agents can be efficiently screened based on the findings discovered by the present inventors.

As described above, the present inventors succeeded in developing anti-rheumatic agents comprising a substance that regulates the autoubiquitination of synoviolin protein as active ingredients, and also developed screening methods for anti-rheumatic agents.

Specifically, the present invention further provides:

[16] an anti-rheumatic agent comprising as an active ingredient a substance having a function of regulating autoubiquitination of a synoviolin protein;
[17] the anti-rheumatic agent of [16], wherein the substance having a function of regulating the autoubiquitination of a synoviolin protein is a low molecular weight compound;
[18] the anti-rheumatic agent of [17], wherein the low molecular weight compound is represented by formula (I) or (II):

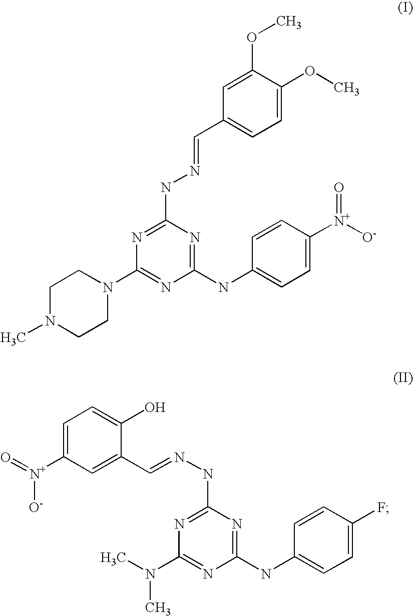

[19] the anti-rheumatic agent of [18], which has an effect of suppressing proliferation of a rheumatoid arthritis synovial cell;
[20] a method of screening for an anti-rheumatic agent, comprising the steps of:

(a) contacting a test compound with a cell expressing a synoviolin gene;

(b) measuring expression level or activity of a synoviolin protein in the cell; and

(c) selecting a compound that lowers the expression level or activity as compared to when the test compound is not contacted;

[21] a method of screening for an anti-rheumatic agent, comprising the steps of:

(a) contacting a test compound with a cell or cell extract which contains DNA having a structure in which a transcriptional regulatory region of a synoviolin gene and a reporter gene are operably linked to each other;

(b) measuring the expression level of the reporter gene; and

(c) selecting a compound that lowers the expression level of the reporter gene as compared to when the test compound is not contacted;

[22] a method of screening for an anti-rheumatic agent, comprising the steps of:

(a) contacting a synoviolin protein and a test compound;

(b) measuring autoubiquitination of the synoviolin protein; and

(c) selecting a compound regulating the autoubiquitination as compared to when the test compound is not contacted; and

[23] a kit for screening for an anti-rheumatic agent, comprising a synoviolin protein as a component.

Furthermore, the present invention provides methods for preventing or treating rheumatic diseases, comprising the step of administering an anti-rheumatic agent of the present invention that comprises a substance having the function of regulating the autoubiquitination of synoviolin protein as an active ingredient, and the use of substances of the present invention that function to regulate autoubiquitination in the production of anti-rheumatic agents.

### BRIEF DESCRIPTION OF THE DRAWINGS

FIG. 1 shows photographs that demonstrate the results of immunofluorescence tissue staining in synoviolin homozygous knockout mouse embryonic fibroblasts (MEFs).

FIG. 2 shows photographs that demonstrate the results of immunohistochemical staining in syno^{-/-} embryos using anti-p53 antibodies.

FIG. 3 shows a photograph that demonstrates the result of Western blotting with respect to p53.

FIG. 4 shows photographs that demonstrate the results of identification of the phosphorylation site of p53 in syno^{-/-} MEF cultured cells.

FIG. 5 shows a photograph of Western blotting in investigating how the addition of caffeine affects p53 Ser15 phosphorylation that had been enhanced by the treatment with synoviolin siRNA.

FIG. 6 shows photographs of Western blotting that demonstrate that the p53 and p21 expressions were enhanced by the treatment with synoviolin siRNA.

FIG. 7 shows graphs that demonstrate the results of observing the cell cycle using a flow cytometer.

FIG. 8 shows photographs that demonstrate the results of immunostaining of tissue arrays using anti-synoviolin antibodies (10 Da).

FIG. 9 shows photographs that demonstrate the results of immunostaining of tissue arrays using anti-synoviolin antibodies (10 Da).

FIG. 10 shows photographs that demonstrate p53 localization in cells into which GFP wild-type p53 had been introduced.

FIG. 11 shows photographs that demonstrate the localizations of the transgene products of both GFP wild-type p53 and FLAG wild-type synoviolin that were coexpressed in Saos-2 cells.

FIG. 12 shows photographs that demonstrate the localizations of transgene products of both GFP wild-type p53 and FLAG synoviolin C307S that were coexpressed in Saos-2 cells.

FIG. 13 shows a photograph that demonstrates the in vitro ubiquitination reaction of GST-p53 by MBP-synoviolin ΔTM-His.

FIG. 14 shows a graph that demonstrates the p53 mRNA level in RA synovial cells by synoviolin RNAi.

FIG. 15 shows a schematic diagram that demonstrates the produced p53-binding domain deletion variants and the results of binding assays.

FIG. 16 shows the results of GST pulldown assays for the p53-binding domain deletion variants and for [³⁵S]-p53.

FIG. 17A shows the formulae of compounds X and Y FIG. 17B shows photographs that demonstrate the inhibitory activities of compounds X and Y on the p53 protein ubiquitination reaction.

FIG. 18 shows the influences of compounds X and Y on cytotoxicity.

FIG. 19 is a photograph that demonstrates the effect of compound Y on the expression level of p53 protein in cultured cells.

FIG. 20 shows the influence of antibody concentration during detection of the in vitro autoubiquitination reaction using ELISA.

FIG. 21 shows the influence of synoviolin concentration on the in vitro autoubiquitination reaction system using ELISA.

FIG. 22 shows the influence of the ubiquitination reaction time on the in vitro autoubiquitination reaction using ELISA.

FIG. 23 shows the inhibitory effect on cell proliferation of low molecular weight compound No. 32.

FIG. 24 shows the inhibitory effect on cell proliferation of low molecular weight compound No. 38.

### BEST MODE FOR CARRYING OUT THE INVENTION

Herein below, the present invention will be explained in detail.

When a normal cell is exposed to ultraviolet rays and such, p53 in the cell is activated, resulting in the inhibition of cell proliferation. Therefore, the proliferation of cancer cells can be inhibited by increasing the p53 concentration. That is, without the function of p53, the proliferation of cancer cells cannot be stopped, and thus cancer progresses. In fact, p53 mutations are rarely found in cells of normal individuals, whereas deletion or point mutations in p53 are found in about half of the cells derived from cancer patients. Moreover, even if such a mutation is not present, other mutations are often found in the p53 control mechanism, and the cancer-suppressing function of p53 is lost. Accordingly, to suppress the progress of cancer, it is essential that p53 functions effectively.

The present inventors focused on the function of synoviolin so as to employ the activation of p53 protein as an effective method for cancer treatment. They produced synoviolin homozygous knockout animals and analyzed them in detail. A large number of cells were observed to be undergoing apoptosis in such animals, as compared with wild-type animals. That is, the present inventors discovered that inhibition of the function of synoviolin promotes the activation of p53 protein, which is deeply involved in apoptosis, and that inhibition of the function of synoviolin leads to cancer suppression.

The present inventors discovered that the inhibition (suppression) of the expression and/or function (activity) of the synoviolin protein leads to the activation of p53 protein. Thus, the present invention provides p53 protein activators comprising, as active ingredients, substances that inhibit the expression and/or function of synoviolin protein.

The species from which the synoviolin protein of the present invention is derived is not particularly limited, but the protein is preferably derived from humans. The term “synoviolin protein” herein includes proteins equivalent to synoviolin (such as synoviolin homologs and orthologs) from organisms other than humans. For example, the present invention can be performed in organisms that have a protein corresponding to human p53 and a protein equivalent to human synoviolin.

Table 1 shows the names and accession numbers of mRNA and amino acid sequences of synoviolin and p53 in humans and “corresponding proteins” in other species.

TABLE 1
p53

| | Standard | mRNA | Protein |
|---|---|---|---|
| Species | nomenclature | accession No. | accession No. |
| human | TP53 | NM_000546 | NP_000537 |
| mouse | Trp53 | NM_011640 | NP_035770 |
| rat | TP53 | NM_030989 | NP_112251 |
| zebrafish | tp53 | NM_131327 | NP_571402 |
| D. melanogaster | p53 CG33336-PA, | NM_206545 | NP_996268 |
| | isoform A | | |
| C. elegans | cep-1 | NM_059861 | NP_492262 |
| human | Synoviolin | AB024690 | BAC57449.1 |
| mouse | Hrd1 | NM_023249 | NP_075738.1 |
| rat | synoviolin 1 | XM_341999 | XP_342000.2 |
| zebrafish | MGC:55735 | BC044465 | AAH44465.1 |
| D. melanogaster | sip3 | NM_143637 | NP_651894.3 |
| C. elegans | 5M249 | NM_073568 | NP_505969.1 |

### Synoviolin

The accession no. of human synoviolin gene in the public gene database Genbank is AB024690 (SEQ ID NO: 1).

The nucleotide sequence of the gene encoding human synoviolin is shown in SEQ ID NO: 1. Moreover, the amino acid sequence of the protein encoded by this nucleotide sequence is shown in SEQ ID NO: 2. Proteins other than the above proteins that are, for example, highly homologous to the sequence in the sequence listing (normally, 70% or more; preferably 80% or more; more preferably 90% or more; and most preferably 95% or more) and that have functions of the synoviolin protein (such as the function of binding with a p53 protein, the function of inhibiting the activity of a p53-phosphorylating protein, and/or the function of promoting the ubiquitination of p53) are included in the synoviolin of the present invention. Examples of these proteins include proteins comprising an amino acid sequence with the addition, deletion, substitution, and/or insertion of one or more amino acids in the amino acid sequence of SEQ ID NO: 2, wherein the number of modified amino acids is normally 30 amino acids or less, preferably 10 amino acids or less, more preferably 5 amino acids or less, and most preferably 3 amino acids or less.

The term “synoviolin gene” as used herein includes, for example, endogenous genes of other organisms that correspond to a DNA comprising the nucleotide sequence of SEQ ID NO: 1 (such as that of homologs of the human synoviolin gene).

Moreover, the endogenous DNA of other organisms that corresponds to DNA comprising the nucleotide sequence of SEQ ID NO: 1 is generally highly homologous to the DNA of SEQ ID NO: 1. The term “highly homologous” means a homology of 50% or more, preferably 70% or more, more preferably 80% or more, and yet more preferably 90% or more (for example, 95% or more, and further, 96%, 97%, 98%, or 99% or more). This homology can be determined using the mBLAST algorithm (Altschul et al., 1990, Proc. Natl. Acad. Sci. USA 87: 2264-8; Karlin and Altschul, 1993, Proc. Natl. Acad. Sci. USA 90: 5873-7). Moreover, if the DNA is isolated from an organism, it is considered to hybridize with the DNA of SEQ ID NO: 1 under stringent conditions. Here, examples of the “stringent conditions” include “2×SSC, 0.1% SDS, 50° C.”, “2×SSC, 0.1% SDS, 42° C.”, and “1×SSC, 0.1% SDS, 37° C.”. Examples of more stringent conditions include “2×SSC, 0.1% SDS, 65° C.”, “0.5×SSC, 0.1% SDS, 42° C.”, and “0.2×SSC, 0.1% SDS, 65° C.”. Those skilled in the art are capable of appropriately obtaining endogenous genes of other organisms that correspond to the synoviolin gene, based on the nucleotide sequence of the synoviolin gene. In the present specification, proteins (genes) corresponding to synoviolin proteins (genes) in organisms other than humans, or proteins (genes) functionally equivalent to the synoviolin described above, may be simply referred to as “synoviolin protein (gene)”.

The “synoviolin protein” of the present invention can be prepared as a natural protein or as a recombinant protein using gene recombination techniques. Natural proteins can be prepared, for example, by a method using affinity chromatography, which employs antibodies against synoviolin protein, from cell (tissue) extracts considered to express the synoviolin protein. In addition, recombinant proteins can be prepared by culturing cells transfected by a DNA encoding the synoviolin protein. The “synoviolin protein” of the present invention is suitably used in, for example, the screening methods described below.

In the present invention, the term “expression” includes “transcription” from genes, “translation” into polypeptides, and the “inhibition of degradation” of proteins. The “expression of synoviolin protein” means the occurrence of the transcription and translation of a gene encoding the synoviolin protein, or the production of the synoviolin protein by such transcription and translation. Moreover, the “function of synoviolin protein” includes, for example, the function of synoviolin in binding with p53, in suppressing the activity of a p53-phosphorylating protein, in promoting the ubiquitination of p53, and in suppressing p53 activation.

The various functions mentioned above can be appropriately evaluated (measured), using general techniques, by those skilled in the art. Specifically, the methods described in the Examples below, or such methods suitably modified, can be performed.

Accordingly, the term “inhibiting the expression and/or function of synoviolin protein” refers to lowering or eliminating the quantity, function, or activity of a synoviolin gene or protein as compared with the quantity, function, or activity of the wild-type synoviolin gene or protein. The term “inhibition” includes the inhibition of either or both of function and expression.

Because synoviolin promotes p53 ubiquitination, the inhibition of the binding between synoviolin and p53 can lead to the inhibition of p53 ubiquitination. Moreover, the inhibition of p53 ubiquitination leads to p53 activation and the suppression of cancer.

In the present invention, the term “p53 protein activators” can more specifically refer to drugs having a function of enhancing (elevating) the expression and/or function of the p53 protein.

The above inhibitory substances of the present invention preferably include low molecular weight compounds. Accordingly, a preferred embodiment of the present invention provides p53 protein activators comprising as an active ingredient a low molecular weight compound that inhibits the expression and/or function of the synoviolin protein.

The present inventors discovered that the inhibition of the expression or function of synoviolin protein leads to inhibition of p53 protein ubiquitination, resulting in activation of the p53 protein.

Synoviolin may promote p53 ubiquitination. The term ubiquitination means the modification reaction of a translated protein by ubiquitin, which is a marker molecule for protein degradation. The physiological significance of ubiquitination has been conventionally recognized as a tag for sending proteins to the degradation mechanism of the proteasome. According to later studies, the significance of ubiquitination, at present, is seen as a reversible protein modification system for controlling the function of proteins.

Specifically, ubiquitination means a process for the formation of a polyubiquitin chain via repeated cascade of reactions with enzymes such as ubiquitin-activating enzyme (E1), ubiquitin-conjugating enzyme (E2), and ubiquitin ligase (E3), by which ubiquitin molecules are conjugated in a branched form to a substrate protein. The polyubiquitin chain is formed via an ε-amino group at Lys48 in ubiquitin molecules, and then becomes a degradation signal for the 26S proteasome, leading to the degradation of target proteins.

The present invention involves the activation of p53 by inhibiting the expression and/or function of synoviolin. This p53 activation is focused on mechanisms of inhibiting p53 ubiquitination rather than the p53 phosphorylation mechanism mentioned above.

In the present invention, low molecular weight compounds that inhibit the expression and/or function of the synoviolin protein preferably have a function of inhibiting p53 protein ubiquitination.

As shown in the Examples below, the present inventors succeeded in identifying low molecular weight compounds (compounds X and Y) that inhibit the activity of synoviolin in promoting p53 ubiquitination.

Specific examples of the low molecular weight compounds of the present invention include compounds represented by the following formula (I) (compound X) and formula (II) (compound Y) identified by the present inventors.

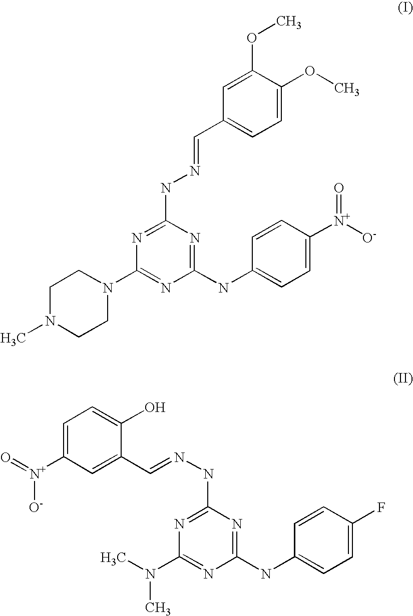

The compounds represented by the above formulae (I) and (II) can be obtained from Namiki Shoji Co., Ltd. (AMBINTER) (the catalog numbers are A0420/0019436 and A1328/0060050, respectively).

Moreover, the compounds of formulae (I) and (II), serving as an active ingredients in the present invention, may be in the form of pharmaceutically acceptable salts of these compounds. Furthermore, hydrates, solvates, and isomers of these compounds are also included in addition to the salts of these compounds. These compounds are also expected to have a p53 activating effect or an anticancer effect.

In the present invention, the “salts” are not specifically limited as long as they form a pharmaceutically acceptable salt with either compound of the present invention; the compounds of formula (I) or (II). Examples of the salts include inorganic acid salts, organic acid salts, inorganic basic salts, organic basic salts, and acidic or basic amino acid salts. Normally, in the present invention, the “salts” generally refer to pharmaceutically acceptable salts.

Preferable examples of the inorganic acid salts include hydrochlorides, hydrobromides, sulfates, nitrates, and phosphates. Preferable examples of the organic acid salts include acetates, succinates, fumarates, maleates, tartrates, citrates, lactates, stearates, benzoates, methanesulfonates, and p-toluenesulfonates.

Preferable examples of the inorganic basic salts include alkali metal salts such as sodium salts and potassium salts, alkaline earth metal salts such as calcium salts and magnesium salts, aluminum salts, and ammonium salts. Preferable examples of the organic basic salts include diethylamine salts, diethanolamine salts, meglumine salts, and N,N′-dibenzylethylenediamine salts.

Preferable examples of the acidic amino acid salts include aspartates and glutamates. Preferable examples of the basic amino acid salts include arginine salts, lysine salts, and ornithine salts.

The compounds of the present invention can be isolated/purified by applying conventional chemical procedures such as extraction, concentration, evaporation, crystallization, filtration, recrystallization, and various forms of chromatography.

In the present invention, the formula of a compound may represent a fixed isomer for the sake of convenience. However, the present invention includes all isomers and mixtures of isomers that occur in the structure of the compound, such as geometric isomers, optical isomers based on asymmetric carbons, stereoisomers, and tautomers. The present invention is not limited to the formulae presented for the sake of convenience, and may be any one of these isomers or a mixture thereof. Accordingly, the compounds of the present invention may have an asymmetric carbon in their molecules, and may exist as optically active substances and racemates. However, the present invention is not limited to these cases, and includes all possible cases.

Moreover, various isomers (such as geometric isomers, optical isomers based on an asymmetric carbon, rotational isomers, stereoisomers, and tautomers) of the compounds of the present invention that can be obtained can be purified and isolated by using common separation means such as recrystallization, diastereomeric salt methods, enzymatic resolution methods, and various kinds of chromatography (for example, thin layer chromatography, column chromatography, gas chromatography, and other forms).

Furthermore, the present inventors discovered that synoviolin has a function of binding to p53 and inhibiting the function of p53. Thus, the inhibition of the expression and/or function of synoviolin leads to the inhibition of the binding between synoviolin and p53, thereby activating the function of p53.

Accordingly, the low molecular weight compounds that inhibit the expression and/or function of the synoviolin protein in the present invention preferably have a function of inhibiting the binding (interaction) between the synoviolin protein and the p53 protein.

The p53-binding domain in the synoviolin protein can be determined, for example, by producing several p53-binding domain deletion variants that lack specific regions in the amino acid sequence of synoviolin, and then performing a GST pulldown assay with [³⁵S]-p53. Specifically, the above p53-binding domain deletion variants of synoviolin are expressed as GST fusion proteins in Escherichia coli or the like, and protein-protein binding of the [³⁵S]-p53 protein is confirmed by the GST pulldown assay.

The above method revealed that the p53-binding domain in synoviolin is 35 amino acid residues from positions 236 to 270 in the amino acid sequence (SEQ ID NO: 2) included in the synoviolin protein.

Accordingly, examples of the above compounds include compounds that inhibit the binding of synoviolin to p53 by targeting the 35-amino acid region from positions 236 to 270 in the synoviolin protein.

Moreover, the present inventors also discovered that inhibition of the expression or function of the synoviolin protein leads to the phosphorylation of some p53 protein amino acid residues by a kinase, thereby activating the p53 protein.

Accordingly, the low molecular weight compounds that inhibit the expression and/or function of the synoviolin protein in the present invention preferably have a function of activating a p53-phosphorylating protein.

The amino acid residue to be subjected to phosphorylation, which leads to p53 protein activation, is preferably a serine residue in the p53 amino acid sequence, and more preferably the serine residue at position 15 (Ser15).

Accordingly, examples of the above compounds can include compounds having a function of increasing the activity of a phosphorylating protein targeting the serine residue at position 15 of p53 as a substrate.

When p53 is phosphorylated at Ser15, p53 expression is elevated and p53 transcription activity is enhanced, resulting in an increase in transcription products. This phosphorylation of p53 at Ser15 is deeply associated with kinases such as ATM (ataxia-telangiectasia mutated) and ATR (ataxia-telangiectasia related). ATM is a causative protein for ataxia-telangiectasia, a human autosomal recessive genetic disease, and ATM has a function of detecting DNA damage and then regulating cell proliferation by phosphorylating the cancer suppressor gene p53. ATR, a member of the ATM family, is a kinase induced by a wide range of chemotherapeutic agents, UV irradiation, or the suppression of protein kinases, and is associated with a form of p53 activation that does not involve ATM.

It is known that the functions of ATM and ATR are inhibited by caffeine. In the present invention, the inhibition experiment for the expression and/or function of synoviolin using caffeine revealed that synoviolin regulates the activation of ATM and ATR.

Specifically, p53 is activated when the expression and/or function of synoviolin are inhibited in the absence of caffeine. On the other hand, when the expression and/or function of synoviolin are inhibited in the presence of caffeine, p53 activation is inhibited. Moreover, it is known that the activities of ATM and ATR (p53 phosphorylation) are inhibited by caffeine.

When the activities of ATM and ATR are inhibited by caffeine, the inhibition of the expression and/or function of synoviolin does not result in the activation of p53. Therefore, a mechanism can be considered in which inhibition of the expression and/or function of synoviolin induces p53 activation by ATM and ATR. Thus, it can be said that inhibition of the expression and/or function of synoviolin leads to increases in the activities of these kinases. Accordingly, the present invention comprises promotion of the activation of kinases by inhibiting the expression and/or function of synoviolin. Enzymes or the like having activities similar to those of ATM and ATR (enzymes that phosphorylate p53) may include DNA-PK, GSK3β, or the like.

Accordingly, in the present invention, the term “p53-phosphorylating protein” is not specifically limited as long as the protein is capable of phosphorylating p53 as a substrate. Normally, the term refers to ATM, ATR, DNA-PK, GSK3β, or the like, and, more preferably, ATM or ATR.

A protein termed p21 is known as a substance whose expression is induced by the phosphorylation of p53 at Ser15. p21 is a protein that functions as an inhibitor of the activity of a cyclin-dependent kinase (CDK), and p21 plays a role in regulating the cell cycle by inhibiting CDK activity. CDK plays a key role in suppressing the cell cycle. CDK functions along with a partner, a cyclin protein, and, for example, governs a smooth transition from G1 phase, which is a dormant cell state, to S phase, which is a DNA replication phase. In cancer cells, as p53 activation leads to increased expression of p21, which is a CDK inhibitor, the transition of cancer cells from G1 phase to S phase is prevented, thereby suppressing the cancer. Accordingly, as described above, the present invention comprises inhibition of the expression and/or function of synoviolin, and thereby the enhancement of p53 activation, leading to induction of p21 expression, and resulting in CDK inhibition to suppress cancer.

The abovementioned low molecular weight compounds of the present invention may be either naturally derived compounds or artificially synthesized compounds. Normally, these compounds can be produced or obtained using methods known to those skilled in the art. Moreover, the compounds of the present invention can also be obtained by screening methods described later.

Furthermore, in the present invention, examples of the substances that inhibit the expression and/or function of synoviolin may include the following compounds (a) and (b):

(a) antibodies that bind to a synoviolin protein and/or p53 protein; and

(b) synoviolin protein mutants having a dominant negative property against the synoviolin protein.

Antibodies that bind to a synoviolin protein and/or a p53 protein can be prepared by methods known to those skilled in the art.

Polyclonal antibodies can be prepared as follows, for example. A small animal such as a rabbit is immunized with a natural synoviolin protein and/or a p53 protein, or a recombinant synoviolin protein and/or a recombinant p53 protein that has been expressed as a GST fusion protein in a microorganism such as E. coli, or a partial peptide thereof, and the animal's serum is obtained. Antibodies are purified from the serum by, for example, ammonium sulfate precipitation, a protein A column, protein G column, DEAE ion exchange chromatography, an affinity column to which the synoviolin protein and/or p53 protein or synthetic peptides are coupled, or the like.

In the case of monoclonal antibodies, for example, a small animal such as a mouse is immunized with the synoviolin protein and/or p53 protein, or partial peptides thereof. Then, the spleen is extracted from the mouse. The spleen is homogenized into separate cells. The cells and mouse myeloma cells are fused using a reagent such as polyethylene glycol. Amongst the fused cells (hybridomas) thus yielded, a clone producing an antibody that binds to the synoviolin protein and/or p53 protein is selected. Next, the obtained hybridoma is transplanted into the abdominal cavity of a mouse, and the ascites fluid is collected from the mouse. The obtained monoclonal antibodies are purified by ammonium sulfate precipitation, a protein A column, a protein G column, DEAE ion exchange chromatography, an affinity column to which the synoviolin protein and/or p53 protein or synthetic peptides are coupled, or the like.

The antibodies of the present invention are not specifically limited as long as they bind to the synoviolin protein and/or the p53 protein of the present invention. Examples of such antibodies include not only polyclonal antibodies and monoclonal antibodies as mentioned above, but also human antibodies, humanized antibodies made by genetic recombination, and antibody fragments or modified antibodies thereof. The antibodies preferably recognize the amino acid region of positions 236-270 in the synoviolin protein, which is involved in the binding with p53. The antibodies are expected to efficiently inhibit the binding between synoviolin and p53.

The synoviolin proteins and/or p53 proteins of the present invention to be used as sensitizing antigens to obtain antibodies are not limited as to their animal origins, but are preferably derived from mammals such as mouse or human, and particularly preferably from a human. Human-derived proteins can be appropriately obtained by those skilled in the art using the gene sequences or the amino acid sequences disclosed herein.

In the present invention, proteins to be used as sensitizing antigens may be complete proteins or partial peptides of proteins. Partial peptides of proteins include, for example, the amino (N)-terminal or carboxy (C)-terminal fragments of proteins. The term “antibody” used herein means an antibody that reacts with the whole length of a protein or with a fragment of the protein.

In addition to obtaining the above hybridomas by immunizing non-human animals with an antigen, hybridomas that produce a desired human antibody having a binding activity to a protein can also be obtained as follows. Human lymphocytes such as those infected with the EB virus are sensitized with a protein, cells expressing the protein, or lysates of such cells, in vitro. Then, the sensitized lymphocytes are fused with human-derived myeloma cells that are capable of indefinite division, such as U266 cells, to yield the desired hybridomas.

Antibodies against the synoviolin protein and/or p53 protein of the present invention are expected to have an effect of inhibiting the expression or function of the synoviolin protein by binding to the synoviolin protein and/or p53 protein, thereby, for example, activating the p53 protein. When an obtained antibody is to be administered to the human body (antibody treatment), it is preferably a human antibody or a humanized antibody to reduce the immunogenicity.

Furthermore, substances capable of inhibiting the expression or function of the synoviolin protein of the present invention include synoviolin protein mutants (synoviolin dominant-negative proteins) having a dominant-negative property against the synoviolin protein. The “synoviolin protein mutants having a dominant-negative property against the synoviolin protein” refer to proteins that function to eliminate or lower the activity of the endogenous wild-type protein when expressed from a gene encoding such a protein. Examples of such synoviolin dominant-negative proteins include synoviolin protein mutants that competitively inhibit the binding of the wild-type synoviolin to p53. More specifically, such proteins include partial peptide fragments of the synoviolin protein that include the p53-binding domain. One of the preferred examples of a dominant-negative protein is a peptide fragment including the amino acid region from positions 236-270 in the amino acid sequence of the synoviolin protein.

In the present invention, examples of the substances that inhibit the expression and/or function of synoviolin protein include substances that inhibit the transcription of the synoviolin gene or translation of the transcription product. In a preferred embodiment of the present invention, the above substances include compounds (nucleic acids) selected from the group consisting of the following (a) to (c):

(a) antisense nucleic acids directed against the transcription product of the synoviolin gene or a portion thereof;

(b) nucleic acids having ribozyme activities that specifically cleaves the transcription product of the synoviolin gene; and

(c) nucleic acids having the effect of inhibiting synoviolin gene expression by an RNAi effect.

The term “nucleic acid” in the present invention means RNA or DNA. Moreover, chemically synthesized nucleic acid analogs such as so-called PNA (peptide nucleic acid) are also included in the nucleic acids of the present invention. PNA is a nucleic acid analog in which the pentose-phosphate backbone, the basic backbone structure of nucleic acids, is replaced with a polyamide backbone composed of glycine units, and has a three-dimensional structure that is very similar to the structure of nucleic acid. Moreover, so-called LNA (locked nucleic acid) is also included in the nucleic acids of the present invention.

To inhibit the expression of a specific endogenous gene, methods utilizing antisense technology are well known to those skilled in the art. There are multiple factors by which an antisense nucleic acid inhibits target gene expression. These include inhibition of transcription initiation by triple strand formation, transcription inhibition by hybrid formation at a local open loop structure formed by RNA polymerase, transcription inhibition by hybrid formation with RNA being synthesized, splicing inhibition by hybrid formation at the junction between an intron and an exon, splicing inhibition by hybrid formation at a spliceosome formation site, inhibition of mRNA translocation from the nucleus to the cytoplasm by hybrid formation with mRNA, splicing inhibition by hybrid formation at a capping site or poly-A addition site, inhibition of translation initiation by hybrid formation at a translation initiation factor-binding site, translation inhibition by hybrid formation at a ribosome binding site near the initiation codon, inhibition of peptide chain elongation by hybrid formation in the translated region or polysome binding site of mRNA, inhibition of gene expression by hybrid formation at a nucleic acid-protein interaction site, etc. As described above, antisense nucleic acids inhibit target gene expression by interfering with various processes such as transcription, splicing, or translation (Hirashima and Inoue, “Shin Seikagaku Jikken Koza” [New Biochemistry Experimentation Lectures] 2; Kakusan (Nucleic Acids) IV; Idenshi No Fukusei To Hatsugen [Replication and Expression of Genes]” Edited by The Japanese Biochemical Society, Tokyo Kagaku Dozin, 319-347, 1993).

The antisense nucleic acids used in the present invention may inhibit synoviolin gene expression and/or function by any of the above mechanisms. In one embodiment, an antisense sequence designed to be complementary to the untranslated region near the 5′-terminal of mRNA of the synoviolin gene is considered to be effective in inhibiting the translation of the gene. Moreover, sequences complementary to the coding region or to the untranslated region on the 3′ side can also be used. Thus, nucleic acids comprising not only antisense sequences of translated regions of the synoviolin gene, but also those of untranslated regions, are included in the antisense nucleic acids used in the present invention. The antisense nucleic acid to be used is linked to the downstream region of an appropriate promoter, and preferably, a sequence containing a transcription termination signal is connected to the 3′ side. A desired animal (cell) can be transformed with the nucleic acid thus prepared using known methods. The sequence of the antisense nucleic acid is preferably complementary to the endogenous-synoviolin gene of the animal (cell) to be transformed or a portion thereof, but the sequence may not be completely complementary as long as the sequence can effectively inhibit gene expression. The transcribed RNA preferably has a complementarity of 90% or more, and most preferably 95% or more, to the transcript of the target gene. In order to effectively inhibit the expression of the target gene (synoviolin) by using an antisense nucleic acid, the antisense nucleic acid is preferably at least 15 bases long but less than 25 bases long. However, antisense nucleic acids of the present invention are not necessarily limited to this length, and may be 100 bases long or longer, or 500 bases long or longer.

Although there is no specific limitation to the antisense nucleic acids used in the present invention, such a nucleic acid could be produced, for example, with reference to the nucleotide sequence of the synoviolin gene (SEQ ID NO: 1) available with GenBank accession No. AB024690.

Moreover, ribozymes or DNAs encoding ribozymes can also be used to inhibit synoviolin gene expression. “Ribozyme” refers to an RNA molecule that has a catalytic activity. Ribozymes have various activities. Studies focusing on ribozymes as RNA cleaving enzymes have made it possible to design ribozymes that site-specifically cleave RNA. Some ribozymes such as group I intron ribozymes or the Ml RNA contained in RNase P consist of 400 nucleotides or more, whereas others, called the hammerhead or hairpin ribozymes, have activity domains of about 40 nucleotides (M. Koizumi and E. Ohtsuka, Tanpakushitsu Kakusan Kohso [Protein, Nucleic Acid, and Enzyme], 35: 2191, 1990).

For example, the self-cleavage domain of hammerhead ribozymes cleaves the 3′ side of C15 in the G13U14C15 sequence. The base pair formation between U14 and A9 is considered important for the above cleavage activity, and it has been shown that the cleavage may also occur when C15 is replaced with A15 or U15 (M. Koizumi et al., FEBS Lett. 228: 228, 1988). When ribozymes are designed to have substrate-binding sites that are complementary to RNA sequences near target sites, they can be restriction enzyme-like RNA-cleaving ribozymes that recognize the sequence of UC, UU, or UA in target RNA (Koizumi, M. et al., FEBS Lett. 239: 285, 1988; M. Koizumi and E. Ohtsuka, Tanpakushitsu Kakusan Kohso [Protein, Nucleic acid, and Enzyme], 35: 2191, 1990; Koizumi, M. et al., Nucl. Acids Res. 17: 7059, 1989).

Hairpin type ribozymes are also useful for the purpose of the present invention. Such a ribozyme can be found, for example, in the minus strand of the satellite RNA of tobacco ringspot virus (Buzayan, J. M., Nature, 323: 349, 1986). It has been shown that target-specific RNA-cleaving ribozymes can also be produced from hairpin type ribozymes (Kikuchi, Y. and Sasaki, N., Nucleic Acids Res. 19: 6751, 1991; Yo Kikuchi, Kagaku To Seibutsu [Chemistry and Biology] 30: 112, 1992). Thus, the expression of the synoviolin gene of the present invention can be inhibited by specifically cleaving the transcript of the gene.

The expression of endogenous genes can also be suppressed by RNA interference (hereinafter abbreviated as “RNAi”) using a double-stranded RNA having the same or similar sequence to the target gene sequence.

Nucleic acids having an inhibitory activity by means of the RNAi effect are generally referred to as siRNA or shRNA. RNAi is a phenomenon in which, when a short double-stranded RNA (herein abbreviated as “dsRNA”) that is composed of a sense RNA comprising a sequence homologous to mRNA of the target gene, and an antisense RNA comprising the complementary sequence thereto, is introduced into a cell, the dsRNA specifically and selectively binds to target gene mRNA and induces its disruption, and efficiently inhibits (suppresses) target gene expression by cleaving the target gene. For example, when a dsRNA is introduced into a cell, the expression of a gene having a sequence homologous to the RNA is suppressed (knocked down). As RNAi is capable of suppressing target gene expression as described above, the technique is attracting attention as a simple gene knockout method replacing conventional gene disruption methods based on homologous recombination, which are complicated and inefficient, and as a method applicable to gene therapy. RNA used for RNAi is not necessarily completely identical to the synoviolin gene or to a partial region of the gene, although it is preferably completely homologous.

siRNA can be designed as follows:

(a) There is no specific limitation to a target region, and any region in the gene encoding synoviolin can be used as a target candidate. For example, in the case of humans, any region in GenBank accession No. AB024690 (SEQ ID NO: 1) can be used as a candidate.

(b) From selected regions, sequences starting with AA, which are 19 to 25 bases long, and preferably 19 to 21 bases long, are selected. For example, sequences having a CG content of 40 to 60% may be selected. Specifically, DNA containing at least one sequence selected from the following nucleotide sequences in the nucleotide sequence shown in SEQ ID NO: 1 can be used as a target sequence of siRNA. In particular, (i) (SEQ ID NO: 3), (ii) (SEQ ID NO: 4), (vi) (SEQ ID NO: 8), (vii) (SEQ ID NO: 9), and (viii) (SEQ ID NO: 10) are preferably used as targets.

| | | |
|---|---|---|
| (i) | AA TGTCTGCATCATCTGCCGA GA | (SEQ ID NO: 3) |
| (ii) | AA GCTGTGACAGATGCCATCA TG | (SEQ ID NO: 4) |
| (iii) | AA AGCTGTGACAGATGCCATC AT | (SEQ ID NO: 5) |
| (iv) | AA GAAAGCTGTGACAGATGCC AT | (SEQ ID NO: 6) |
| (v) | AA GGTTCTGCTGTACATGGCC TT | (SEQ ID NO: 7) |
| (vi) | AA CAAGGCTGTGTACATGCTC TA | (SEQ ID NO: 8) |
| (vii) | AA ATGTTTCCACTGGCTGGCT GA | (SEQ ID NO: 9) |
| (viii) | AA GGTGTTCTTTGGGCAACTG AG | (SEQ ID NO: 10) |
| (ix) | AA CATCCACACACTGCTGGAC GC | (SEQ ID NO: 11) |
| (x) | AA CACCCTGTATCCAGATGCC AC | (SEQ ID NO: 12) |
| (xi) | AA GGTGCACACCTTCCCACTC TT | (SEQ ID NO: 13) |
| (xii) | AA TGTTTCCACTGGCTGGCTG AG | (SEQ ID NO: 14) |
| (xiii) | AA GAGACTGCCCTGCAACCAC AT | (SEQ ID NO: 15) |
| (xiv) | AA CGTTCCTGGTACGCCGTCA CA | (SEQ ID NO: 16) |

In order to introduce siRNA into cells, methods in which siRNA synthesized in vitro is linked to a plasmid DNA and then introduced into cells, and methods in which two RNAs are annealed, and the like, may be employed.

Moreover, a preferable example of an siRNA of the present invention is a double-stranded RNA produced by annealing the sense strand of SEQ ID NO: 18 and the antisense strand of SEQ ID NO: 19. More specifically, this RNA is an RNA molecule having the following structure.

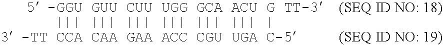

(“|” denotes a hydrogen bond.)

The above RNA molecule may be a molecule in which either end is closed, such as an siRNA having a hairpin structure (shRNA). shRNA is called a short hairpin RNA, which is a RNA molecule having a stem-loop structure because a portion of a single strand forms a complementary strand with another region. Thus, molecules capable of forming an intramolecular double-stranded RNA structure are also included in the siRNA of the present invention.

Moreover, in a preferred embodiment of the present invention, the siRNA of the present invention also includes siRNAs that target any one of the DNA sequences specifically shown in the present specification (SEQ ID NOs: 3 to 16) and that can suppress the synoviolin gene expression by the RNAi effect, as well as double-stranded RNAs of a structure in which one or a small number of RNAs are added to or deleted from the siRNA nucleotide sequence formed by SEQ ID NOs: 18 and 19, as long as these siRNAs retain the function of suppressing synoviolin gene expression.

The RNA used for RNAi (siRNA) may not be necessarily completely identical (homologous) to the synoviolin gene or to a partial region of the gene, although the RNA is preferably completely identical (homologous).

Although some details of the RNAi mechanism are still unknown, it is considered that an enzyme called DICER (a member of the RNase III nuclease family) comes into contact with a double-stranded RNA and decomposes it into small fragments called small interfering RNAs or siRNAs. Double-stranded RNAs having the RNAi effect in the present invention also include double-stranded RNAs prior to being decomposed by DICER as described above. Thus, the length of double-stranded RNAs in the present invention is not specifically limited, because even long-chain RNAs that do not have the RNAi effect originally are expected to be decomposed into siRNAs having the RNAi effect in cells.

For example, it is possible to decompose, in advance, a long-chain double-stranded RNA corresponding to a full-length or almost full-length region of the synoviolin gene mRNA of the present invention with DICER, and then to use the decomposed products as agents of the present invention. The decomposed products are expected to contain double-stranded RNA molecules having the RNAi effect (siRNAs). By this method, it is not necessary to select a particular mRNA region that is expected to have the RNAi effect. Thus, there is no need to accurately define the mRNA region of the synoviolin gene of the present invention having the RNAi effect.

The “double-stranded RNA having the RNAi effect” of the present invention can be appropriately produced by those skilled in the art based on the nucleotide sequence of the synoviolin gene of the present invention serving as a target of the double-stranded RNA. For example, the double-stranded RNA of the present invention can be produced based on the nucleotide sequence of SEQ ID NO: 1. Thus, it is within the common practice of those skilled in the art to appropriately select, based on the nucleotide sequence of SEQ ID NO: 1, an arbitrary continuous RNA region in the mRNA transcribed from the sequence, and to produce double-stranded RNA corresponding to this region. Moreover, those skilled in the art can also appropriately select an siRNA sequence having a stronger RNAi effect from the mRNA sequence transcribed from the sequence, using known methods. Furthermore, if the sequence of one strand (for example, the nucleotide sequence of SEQ ID NO: 1) is known, the nucleotide sequence of the other strand (complementary strand) can be readily identified by those skilled in the art. siRNA can be appropriately produced by those skilled in the art using a commercially available nucleic acid synthesizer. Moreover, desired RNAs can be synthesized by using general custom synthesis services.

Furthermore, the nucleotides of the siRNA of the present invention may not necessarily all consist of ribonucleotides (RNA). Specifically, in the present invention, one or more ribonucleotides constituting siRNA may be corresponding deoxyribonucleotides. The term “corresponding” refers to possession of the same base (adenine, guanine, cytosine, and thymine (uracil)) although with a different sugar structure. For example, a deoxyribonucleotide corresponding to a ribonucleotide with adenine refers to a deoxyribonucleotide with adenine. Moreover, the above term “more” is not specifically limited, but preferably refers to a small number of about two to five.

Furthermore, DNAs (vectors) capable of expressing the RNAs of the present invention are also included in a preferred embodiment of the compounds capable of suppressing the synoviolin gene expression of the present invention. For example, DNAs (vectors) capable of expressing the double-stranded RNAs of the present invention have a structure in which a DNA encoding one strand of the double-stranded RNA and a DNA encoding the other strand of the double-stranded RNA are associated with promoters so that both DNAs can be expressed. These DNAs of the present invention can be appropriately produced by those skilled in the art using common genetic engineering techniques. More specifically, the expression vectors of the present invention can be produced by appropriately inserting a DNA encoding an RNA of the present invention into various known expression vectors.

Furthermore, DNAs (vectors) capable of expressing the RNAs of the present invention are also included in a preferred embodiment of the compounds capable of suppressing the synoviolin gene expression of the present invention. For example, DNAs (vectors) capable of expressing the double-stranded RNAs of the present invention have a structure in which a DNA encoding one strand of the double-stranded RNA and a DNA encoding the other strand of the double-stranded RNA are associated with promoters so that both DNAs can be expressed. These DNAs of the present invention can be appropriately produced by those skilled in the art using common genetic engineering techniques. More specifically, the expression vectors of the present invention can be produced by appropriately inserting a DNA encoding an RNA of the present invention into various known expression vectors.

Moreover, the substances that inhibit synoviolin expression of the present invention include compounds that inhibit synoviolin expression by binding to an expression regulatory region for synoviolin (such as a promoter region). These compounds can be obtained by, for example, a screening method in which a DNA fragment of a promoter for synoviolin is used and the binding activity with the DNA fragment is used as an index. Moreover, those skilled in the art are capable of appropriately determining whether or not a desired compound inhibits the synoviolin expression by known methods such as the reporter assay method.

Furthermore, DNAs (vectors) capable of expressing the RNAs of the present invention are also included in a preferred embodiment of the compounds capable of suppressing the synoviolin gene expression of the present invention. For example, DNAs (vectors) capable of expressing the double-stranded RNAs of the present invention have a structure in which a DNA encoding one strand of the double-stranded RNA and a DNA encoding the other strand of the double-stranded RNA are associated with promoters so that both DNAs can be expressed. These DNAs of the present invention can be appropriately produced by those skilled in the art using common genetic engineering techniques. More specifically, the expression vectors of the present invention can be produced by appropriately inserting a DNA encoding an RNA of the present invention into various known expression vectors.

Preferred embodiments of the vectors of the present invention may include vectors that express siRNAs that inhibit synoviolin expression.

Moreover, the substances that inhibit the synoviolin expression of the present invention include compounds that inhibit the synoviolin expression by binding to an expression regulatory region for synoviolin (such as a promoter region). These compounds can be obtained by, for example, a screening method in which a DNA fragment of a promoter for synoviolin is used and the binding activity with the DNA fragment is used as an index. Moreover, those skilled in the art are capable of appropriately determining whether or not a desired compound inhibits the synoviolin expression by known methods such as the reporter assay method.

The p53 protein-activating agents of the present invention have an effect of inhibiting the proliferation of cancer cells by activating p53, which is a cancer suppressor protein. Accordingly, the present invention provides cancer therapeutic agents comprising the p53 protein-activating agents of the present invention as active ingredients.

It is considered that the inhibition of the expression or function of synoviolin activates p53 because of the phosphorylation of p53 by a kinase, which in turn enhances the expression of p21 protein, an inhibitory protein of a cyclin-dependent kinase. This results in the interference with the transition from G1 phase to S phase of tumor cells, thereby inhibiting the development or the proliferation of cancer. Accordingly, drugs comprising the p53 protein-activating agents of the present invention can be agents for activating p21 protein or agents for inhibiting cell cycle progression (in particular, inhibiting the transition from G1 phase to S phase).

In addition to cancer as mentioned above, examples of known diseases in which p53 is involved also include neurodegenerative diseases (Jikken Igaku [Experimental medicine], Zokango (extra number), 2001, p53; Kenkynuno Aratana Tyousen [New Challenges for p53 Research] p. 122-127). It is therefore considered that the p53 protein-activating agents of the present invention can also be agents for treating neurodegenerative diseases.

The term “cancer therapeutic agents” of the present invention may also be referred to as “agents for inhibiting cancer cell proliferation”, “agents for inhibiting cancer cell development”, “agents for inhibiting tumor proliferation”, “anticancer agents”, or “carcinostatic agents”. Moreover, in the present invention, the term “therapeutic agents” may also be referred to as “pharmaceuticals”, “pharmaceutical compositions”, “therapeutic medicines”, or the like.

The term “treatment/therapy” in the present invention also includes preventive effects capable of suppressing the development of cancer beforehand. Moreover, the “treatment/therapy” may not necessarily show complete therapeutic effects on cancer cells (tissues), but may show partial effects.

The agents of the present invention can be combined with a physiologically acceptable carrier, excipient, diluent, or the like, and then administered orally or parenterally as pharmaceutical compositions. The dosage form for oral agents may be granules, powders, tablets, capsules, solutions, emulsions, suspensions, or the like. The dosage form for parenteral agents may be selected from injections, drops, external medicines, suppositories, or the like. Examples of injections include agents for subcutaneous injection, intramuscular injection, and intraperitoneal injection. Examples of external medicines include agents for nasal administration and ointments. Preparation methods for preparing the above dosage forms to include agents of the present invention as the main ingredients are known.

For example, tablets for oral administration can be produced by adding an excipient, a disintegrator, a binder, a lubricant, and the like, to the agents of the present invention, followed by mixing and compression shaping. Lactose, starch, mannitol, and the like are commonly used as excipients. Calcium carbonate, carboxymethylcellulose calcium, and the like, are commonly used as disintegrators. Gum Arabic, carboxymethylcellulose, or polyvinylpyrrolidone, are used as binders. Talc, magnesium stearate, and the like, are used as lubricants.

Tablets including the agents of the present invention can be masked or coated to form enteric preparations by known methods. Ethylcellulose, polyoxyethyleneglycol, and the like may be used as coating agents.

Moreover, injectable preparations can be obtained by mixing the agents of the present invention, serving as the main ingredients, with an appropriate dispersing agent, or by dissolving or dispersing them in a dispersion medium. The agents may be in the form of either an aqueous preparation or an oil-based preparation by appropriate selection of dispersion medium. Distilled water, physiological saline, Ringer's solution, or the like is used as dispersion media when preparing aqueous preparations. Various vegetable oils, propylene glycol, or the like, is used as dispersion media for oil-based preparations. Preservatives such as paraben may also be added as required. Moreover, publicly known isotonizing agents such as sodium chloride and glucose can be added to the injectable preparations. Furthermore, soothing agents such as benzalkonium chloride and procaine hydrochloride can be added.

Moreover, external preparations can be produced by forming the agents of the present invention into solid, liquid, or semisolid compositions. In the case of solid or liquid compositions, external preparations can be produced by making compositions similar to those described above. Semisolid compositions can be prepared by adding thickeners to appropriate solvents as required. Water, ethyl alcohol, polyethylene glycol, or the like can be used as solvents. Bentonite, polyvinyl alcohol, acrylic acid, methacrylic acid, polyvinylpyrrolidone, or the like are commonly used as thickeners. Preservatives such as benzalkonium chloride can be added to these compositions. Moreover, these compositions can also be combined with oil bases such as cacao butter or with aqueous gel bases such as cellulose derivatives as carriers, to prepare suppositories.

If the agents of the present invention are used as agents for gene therapy, there are methods of directly administering the agents of the present invention by injection, and methods of administering vectors incorporating a nucleic acid. Examples of the vectors include adenoviral vectors, adeno-associated viral vectors, herpesvirus vectors, vaccinia virus vectors, retroviral vectors, and lentiviral vectors. The use of such viral vectors enables efficient administration of therapeutic agents.

Moreover, it is possible to introduce the agents of the present invention into phospholipid vesicles such as liposomes and then to administer the vesicles. Vesicles retaining siRNA or shRNA are introduced into given cells by the lipofection method. Cells thus obtained are systemically administered into a vein or an artery, or the like. The cells can also be locally administered into a cancer tissue or the like.

Agents of the present invention are administered into mammals including humans at necessary amounts (effective amounts), within ranges regarded as safe doses. The dose of an agent of the present invention can be suitably determined by consideration of the dosage form, the administration method, the age and body weight of the patient, the symptoms of the patient, and the like, and finally by the judgment of doctors or veterinarians. For example, the dose of adenovirus is about 10⁶ to 10¹³ once a day, administered at intervals of 1-8 weeks, although the dose varies with age, gender, symptoms, administration route, frequency of administration, and dosage form.

In addition, to introduce siRNA or shRNA into desired tissues or organs, commercially available kits for gene introduction (such as AdenoExpress [Clontech]) can be used.

Agents of the present invention can be applied to, for example, brain tumors, tongue cancer, pharyngeal cancer, lung cancer, breast cancer, esophageal cancer, stomach cancer, pancreatic cancer, biliary tract cancer, gallbladder cancer, duodenal cancer, colon cancer, liver cancer, uterine cancer, ovarian cancer, prostate cancer, renal cancer, bladder cancer, rhabdomyosarcoma, fibrosarcoma, osteosarcoma, chondrosarcoma, skin cancer, and various leukemias (such as acute myeloid leukemia, acute lymphoid leukemia, chronic myeloid leukemia, chronic lymphoid leukemia, adult T-cell leukemia, and malignant lymphoma), and the site of agent application, and type of cancer, is not particularly limited. The above cancers may be either primary or metastasized cancers, or may present in the form of a complication with other diseases.

Moreover, the present invention provides methods of screening for cancer therapeutic agents, using the synoviolin gene expression level as an index.

Compounds that lower (suppress) synoviolin gene expression are expected to be drugs for cancer treatment. According to the screening methods of the present invention, cancer therapeutic agents or candidate compounds for cancer therapeutic agents can be efficiently obtained.

A preferred embodiment of the method of the present invention is a method of screening for cancer therapeutic agents comprising the following steps (a) to (c):

(a) contacting a test compound with a cell expressing the synoviolin gene;

(b) measuring synoviolin gene expression level in the cell; and

(c) selecting a compound lowering the expression level as compared to that measured in the absence of the test compound.

In the above method, first, a test compound is contacted with cells expressing the synoviolin gene. The “cells” to be used include those derived from a human, mouse, rat, or the like, but are not particularly limited to these cells. Microbial cells such as E. coli and yeast, transformed to express synoviolin, can also be used. The “cells expressing the synoviolin gene” may be either cells expressing an endogenous synoviolin gene, or cells expressing a foreign synoviolin gene introduced into the cells. Typically, the cells expressing a foreign synoviolin gene can be produced by introducing an expression vector having the synoviolin gene inserted therein into a host cell. The expression vector can be produced with common genetic engineering techniques.

The test compound to be used in the present method is not specifically limited, but includes, for example, single compounds such as natural compounds, organic compounds, inorganic compounds, proteins, and peptides, as well as compound libraries, expression products from gene libraries, cell extracts, cell culture supernatants, microbial fermentation products, marine organism extracts, plant extracts, and the like.

The “contact” of the test compound with the cell expressing the synoviolin gene is normally performed by adding the test compound to a culture fluid containing cells expressing the synoviolin gene, although it is not limited to this method. If the test compound is a protein or the like, the “contact” can be performed by introducing a DNA vector expressing the protein into the cell.

In the present method, the synoviolin gene expression level is then measured. Herein, the term “gene expression” includes both gene transcription and translation. The gene expression level can be measured with methods known to those skilled in the art.

For example, the gene transcription level can be measured by extracting mRNA from cells expressing the synoviolin gene according to a usual method, and performing Northern hybridization, RT-PCR, a DNA array assay method, and the like, using the mRNA as a template. Moreover, the gene translation level can be measured by collecting a protein fraction from cells expressing the synoviolin gene, and by then detecting synoviolin protein expression using electrophoretic methods such as SDS-PAGE. Furthermore, the gene translation level can also be measured by conducting Western blotting using an antibody against the synoviolin protein to detect protein expression. The antibody used for detecting the synoviolin protein is not specifically limited as long as the antibody can detect the protein, and, for example, both monoclonal antibodies and polyclonal antibodies can be used.

In the present method, compounds that lower synoviolin expression level as compared to the situation where the test compound is not contacted (control) are selected. The compounds lowering the expression level of synoviolin serve as drugs for treating cancer.

Another embodiment of the screening method of the present invention is a method using the activity of the synoviolin protein as an index. The method is, for example, a method of screening for cancer therapeutic agents comprising the following steps (a) to (c):

(a) contacting a test compound with a synoviolin protein, a cell expressing the synoviolin protein, or an extract of such a cell;

(b) measuring the activity of the synoviolin protein; and

(c) selecting a compound lowering the protein activity as compared with the situation where the test compound is not contacted.

In the present method, first, a test compound is contacted with a synoviolin protein, a cell expressing the protein, or an extract of such a cell.

Next, the activity of the synoviolin protein is measured. Examples of synoviolin protein activities include the binding activity (interaction activity) with the p53 protein, the activity of promoting the ubiquitination of p53 protein, and the activity of inhibiting the activation of a p53-phosphorylating protein. These activities can be appropriately measured by those skilled in the art, using known methods, such as immunoprecipitation, pulldown assays, and the yeast two-hybrid method.

Furthermore, compounds lowering (suppressing) synoviolin protein activity as compared to the situation where the test compound is not contacted (control) are selected. The compounds lowering (suppressing) the synoviolin protein activity serve as drugs for treating cancer.

Another embodiment of the screening method of the present invention is a method in which compounds lowering the expression level of the synoviolin gene of the present invention are selected using the expression of a reporter gene as an index.

A preferred embodiment of the above method of the present invention is a method of screening for cancer therapeutic agents comprising the following steps (a) to (c):

(a) contacting a test compound with a cell or cell extract that contains DNA of a structure in which the transcriptional regulatory region of the synoviolin gene and a reporter gene are operably linked to each other;

(b) measuring the expression level of the reporter gene; and

(c) selecting a compound lowering the reporter gene expression level as compared to the level seen in the absence of contact of the test compound.

In the present method, first, a test compound is contacted with cells containing DNA of a structure in which the transcriptional regulatory region of the synoviolin gene and a reporter gene are operably linked to each other, or to an extract of such cells. Herein, the term “operably linked to each other” refers to the transcriptional regulatory region of the synoviolin gene and the reporter gene; these are linked so that the expression of the reporter gene is induced by the binding of transcription factors to the transcriptional regulatory region of the synoviolin gene. Accordingly, the meaning of the term “operably linked” also includes the cases where a reporter gene is linked to another gene to form a fusion protein with a product of the other gene, as long as the expression of the fusion protein is induced by the binding of transcription factors to the transcriptional regulatory region of the synoviolin gene. The transcriptional regulatory region of the synoviolin gene in the genome can be obtained by those skilled in the art using known methods, based on the cDNA nucleotide sequence of the synoviolin gene.

The reporter gene used in the present method is not specifically limited, as long as gene expression is detectable. Examples of reporter genes include the CAT gene, the lacZ gene, the luciferase gene, and the GFP gene. The “cells that contain DNA of a structure in which the transcriptional regulatory region of the synoviolin gene and a reporter gene are operably linked to each other” include, for example, cells into which a vector containing such a structure have been introduced. Such vectors can be produced using methods known to those skilled in the art. Vectors can be introduced into cells using common methods, such as the calcium phosphate precipitation method, the electroporation method, the lipofection method, and the microinjection method. The “cells that contain DNA of a structure in which the transcriptional regulatory region of the synoviolin gene and a reporter gene are operably linked to each other” also include cells in which the structure has been inserted in the chromosome. The DNA structure can be inserted into the chromosome by methods usually employed by those skilled in the art, such as gene introduction methods using homologous recombination.

The “cell extract that contains DNA of a structure in which the transcriptional regulatory region of the synoviolin gene and a reporter gene are operably linked to each other” includes, for example, cell extracts contained in commercially available kits for in vitro transcription and translation, to which the DNA of a structure in which the transcriptional regulatory region of the synoviolin gene and the reporter gene are operably linked to each other is added.

In the present method, the “contact” can be performed by adding the test compound to a culture fluid of “cells that contain DNA of a structure in which the transcriptional regulatory region of the synoviolin gene and a reporter gene are operably linked to each other”, or by adding the test compound to a commercially available cell extract containing such DNA. When the test compound is a protein, the “contact” can be performed by introducing a DNA vector expressing the protein into the cells.

In the present method, the expression level of the reporter gene is then measured. The reporter gene expression level can be measured by methods known to those skilled in the art depending on the type of the reporter gene. For example, when the reporter gene is a CAT gene, the reporter gene expression level can be measured by detecting the acetylation of chloramphenicol by the product of the gene. When the reporter gene is a lacZ gene, luciferase gene, or GFP gene, the reporter gene expression level can be measured by detecting the color development of a pigmented compound arising from the catalytic effect of the LacZ gene expression product; the fluorescence of a fluorescent compound arising from the catalytic effect of luciferase; or the fluorescence of the GFP protein, respectively.

In the present method, compounds that lower (suppress) the measured expression level of the reporter gene as compared with the level measured in the absence of the test compound (control) are selected. The compounds lowering (suppressing) the expression level serve as drugs for treating cancer, or as candidate compounds for treating cancer.

Another embodiment of the screening method of the present invention is a method using the binding activity between the synoviolin protein and the p53 protein as an index. The synoviolin protein of the present invention has a binding (interaction) activity with the p53 protein. Accordingly, using the binding activity between the synoviolin protein and the p53 protein as an index, drugs for treating cancer can be screened by selecting substances lowering (suppressing) the binding activity.

For example, the above method of the present invention is a method of screening for cancer therapeutic agents comprising the following steps (a) to (c):

(a) contacting a synoviolin protein, a p53 protein, and a test compound;

(b) measuring the binding activity between the synoviolin protein and the p53 protein; and

(c) selecting a compound lowering the binding activity as compared to when the test compound is not contacted.

In the above method, first, a synoviolin protein, a p53 protein, and a test compound are contacted. Next, the binding activity between the synoviolin protein and the p53 protein is measured.

Normally, the binding (interaction) activity between the synoviolin protein and the p53 protein can be readily measured by those skilled in the art. The measurement of the binding activity in the above step (b) can be appropriately performed by employing common methods such as immunoprecipitation or pulldown assay.

The synoviolin protein and the p53 protein used in the above method are preferably wild-type proteins without mutation, although they may also be proteins or polypeptides with substitution or deletion of some of the amino acids of these wild-type proteins, as long as the binding (interaction) activity is retained.

The synoviolin protein used in the above method is preferably a wild-type protein (full-length protein) without mutation, although it may be a partial peptide fragment thereof or a protein or polypeptide with substitution or deletion of some amino acids in the wild-type protein, as long as the binding activity with the p53 protein is retained.

The region in the synoviolin protein involved in binding with the p53 protein is position 236-270 in the amino acid sequence of the synoviolin protein (SEQ ID NO: 2). Accordingly, polypeptide fragments of the synoviolin protein containing the amino acid region from position 236-270 can be suitably used in the present screening method.

In the above method, compounds lowering (suppressing) the binding activity as compared with the situation where the test compound is not contacted (control) are selected. The compounds lowering (suppressing) the binding activity serve as drugs for treating cancer.

Another embodiment of the screening method of the present invention includes a method using the ubiquitination of p53 protein as an index. The synoviolin protein of the present invention has a function of promoting p53 protein ubiquitination. Accordingly, using p53 protein ubiquitination as an index, drugs for treating cancer can be screened by selecting a substance lowering (suppressing) the ubiquitination.

For example, the above method of the present invention is a screening method for cancer therapeutic agents comprising the following steps (a) to (c):

(a) contacting a synoviolin protein, a p53 protein, and a test compound;

(b) measuring the ubiquitination of p53 protein; and

(c) selecting a compound lowering ubiquitination as compared to when the test compound is not contacted.

In the above method, first, a synoviolin protein, a p53 protein, and a test compound are contacted. Next, the degree of the ubiquitination of p53 protein is measured.

The degree of the p53 protein ubiquitination can be measured with common methods by those skilled in the art.

In one example, the in vitro ubiquitination reaction is measured using GST-p53 and MBP-Synoviolin ΔTM-His. First, GST-p53 is purified from an E. coli (BL21) extract containing pGEX/p53 using a GSH-Sepharose resin. The reaction is carried out using the dialyzed sample in combination with MBP-Synoviolin ΔTM-His and other compositions (ATP, PK-His-HA-Ub, yeast E1, and His-UbCH5c) used in the in vitro ubiquitination reaction. After the reaction, the proteins are separated by SDS-PAGE and transferred to a PVDF membrane. Then, the p53 protein on the membrane is detected with an anti-p53 antibody (FL393 or DO-1), by which means in vitro ubiquitination can be measured.

In the above method, next, compounds lowering (suppressing) the degree of ubiquitination as compared to when the test compound is not contacted (control) are selected. The compounds lowering (suppressing) the degree of ubiquitination serve as drugs for treating cancer, or candidate compounds for treating cancer.

Another embodiment of the screening method of the present invention includes a method using, as an index, phosphorylation activity of the p53 phosphorylating protein that targets the p53 protein as a substrate. The synoviolin protein inhibits p53 activity by inhibiting the phosphorylating activity of the p53-phosphorylating protein. Accordingly, substances increasing the activity of the p53-phosphorylating protein are expected to have a function of activating p53.

The present invention is a method of screening for cancer therapeutic agents comprising the following steps (a) to (c):

(a) contacting a synoviolin protein, a p53 protein, a p53-phosphorylating protein, and a test compound;

(b) measuring the phosphorylating activity of the p53 phosphorylating protein that targets the p53 protein as a substrate; and

(c) selecting a compound increasing the phosphorylating activity as compared to when the test compound is not contacted.

In the above method, first, a synoviolin protein, a p53 protein, and a test compound are contacted. Next, the phosphorylating activity of the p53 phosphorylating protein that targets the p53 protein as a substrate, is measured.

As described above, the “p53-phosphorylating protein” in the present invention is not specifically limited as long as it is a protein capable of phosphorylating p53 as a substrate, although the term normally refers to ATM, ATR, DNA-PK, GSK3β, or the like, and more preferably ATM or ATR.

The p53-phosphorylating protein used in the above method is preferably a wild-type protein without mutation, although it may also be a protein (polypeptide) having a substitution or deletion of some amino acids in the p53 phosphorylating protein as long as it retains the activity of phosphorylating the p53 protein.

Moreover, the p53 protein used as a substrate is also preferably a wild-type protein (full-length protein) without mutation, although it may also be a partial peptide fragment (fragment peptide) thereof or a protein (polypeptide) having a substitution of or deletion of a part of the amino acid sequence of the protein, as long as the resulting protein can be phosphorylated by the p53-phosphorylating protein. More preferably, the protein may be a partial peptide or a mutant polypeptide containing a site to be phosphorylated.

In the above method, the site in the p53 protein to be phosphorylated by the p53-phosphorylating protein is not specifically limited, although it is preferably a serine residue at position 15 in the amino acid sequence of the p53 protein (such as the amino acid sequence of SEQ ID NO: 17). Accordingly, in the present method, a polypeptide fragment of p53 containing the serine residue at position 15 can be suitably used.

As described above, the kinase activity of the p53-phosphorylating protein can be measured by, for example, the Western blotting method, using a phosphorylation-specific antibody.

In the above method, furthermore, compounds are selected that increase the phosphorylating activity of the p53-phosphorylating protein, as compared to when the test compound is not contacted. Normally, such compounds can be selected using the phosphorylation state of the p53 protein or the peptide fragment of p53 as an index. Thus, selected compounds are expected to promote the phosphorylating activity of the p53-phosphorylating protein that targets p53 protein as a substrate, resulting in inhibition of cancer cell proliferation, thereby exerting a cancer therapeutic effect.

Moreover, the present invention provides kits comprising various agents/reagents and the like to be used in the performance of the screening methods of the present invention.

As for the kits of the present invention, for example, appropriate reagents can be selected from the various abovementioned reagents of the present invention, depending on the screening method to be performed. For example, the kits of the present invention may include at least (a) a synoviolin protein and (b) a p53 protein of the present invention as components. The kits of the present invention may further include various reagents, containers, and the like, to be used for the method of the present invention. For example, the kits may appropriately contain anti-synoviolin antibodies, anti-phosphorylated-Ser antibodies, probes, various reaction reagents, cells, culture media, control samples, buffers, instructions describing how to use the kit, and the like.

A preferred embodiment of the present invention is a method of screening for cancer therapeutic agents that includes the step of detecting whether or not the expression and/or function of the synoviolin protein is inhibited. Accordingly, oligonucleotides such as probes for the synoviolin gene and primers for amplifying an arbitrary region of the gene, and antibodies recognizing the synoviolin protein (anti-synoviolin protein antibodies), which can be used for detecting the synoviolin protein in the screening method, may also be included as components of the kits of the present invention for screening for cancer therapeutic agents.

These oligonucleotides specifically hybridize with DNA of the synoviolin gene of the present invention, for example. Herein, the term “specifically hybridize” means that the oligonucleotides do not significantly cross-hybridize with DNA encoding other proteins under normal hybridization conditions, and preferably stringent hybridization conditions (for example, conditions described in Sambrook et al., Molecular Cloning, Cold Spring Harbor Laboratory Press, New York, USA, Second Edition, 1989). The oligonucleotides do not need to be completely complementary to the nucleotide sequence of the synoviolin gene as long as specific hybridization is possible.

In the present invention, the hybridization conditions include, for example, “2×SSC, 0.1% SDS at 50° C.”, “2×SSC, 0.1% SDS at 42° C.”, and “1×SSC, 0.1% SDS at 37° C.”. More stringent conditions include “2×SSC, 0.1% SDS at 65° C.”, “0.5×SSC, 0.1% SDS at 42° C.”, and “0.2×SSC, 0.1% SDS at 65° C.”. More specifically, a method using Rapid-Hyb buffer (Amersham Life Sciences) can be conducted as follows: prehybridization is performed at 68° C. for 30 min or more, then a probe is added and allowed to hybridize at 68° C. for 1 h or more. Then, washing is carried out three times with 2×SSC, 0.1% SDS at room temperature for 20 min, then three times with 1×SSC, 0.1% SDS at 37° C. for 20 min, and finally twice with 1×SSC, 0.1% SDS at 50° C. for 20 min. Otherwise, the following procedure can also be conducted, for example: prehybridization is performed in Expresshyb Hybridization Solution (Clontech) at 55° C. for 30 min or more, then a labeled probe is added and incubated at 37-55° C. for 1 h or more. Then washing is carried out three times with 2×SSC, 0.1% SDS at room temperature for 20 min, and once with 1×SSC, 0.1% SDS at 37° C. for 20 min. Here, more stringent conditions can be applied by, for example, setting higher temperatures for the prehybridization, the hybridization, and the second washing. For example, temperatures for the prehybridization and the hybridization may be set at 60° C., and, to increase stringency even further, the temperatures may be set to 68° C. Those skilled in the art can set appropriate conditions by taking into consideration other conditions such as the probe concentration, the probe length, the nucleotide sequence composition of the probe, and the reaction time, in addition to the above conditions pertaining to the salt concentration in the buffer, the temperature, and the like.

The oligonucleotides can be used as probes or primers in the screening kits of the present invention. When the oligonucleotides are used as primers, their length is normally 15-100 bp, and preferably 17-30 bp. The primers are not specifically limited as long as they can amplify at least a part of DNA of the synoviolin gene of the present invention.

In the present invention, the immunostaining of p53 in fetal embryos showed that p53 was strongly expressed systemically in embryos of synoviolin homozygous knockout mice. Moreover, mouse embryonic fibroblasts (MEFs) isolated from the embryos of the synoviolin homozygous knockout mice showed stronger p53 expression than did those isolated from wild-type mice, and also showed strong nuclear localization of p53. No nuclear localization was seen in wild-type mice. Moreover, when synoviolin and p53 were overexpressed, p53 colocalized with synoviolin in the cytoplasm. This means that inhibition of synoviolin expression and/or function enables the translocation of p53 into the nucleus.

Furthermore, MEFs of the synoviolin homozygous knockout mouse embryos showed high sensitivity to radiation or UV irradiation. Accordingly, in the present invention, cancer cell proliferation can be efficiently suppressed by inhibiting synoviolin expression and/or function in cancer cells, to permit translocation of p53 into the nuclei of the cancer cells, and by then irradiating the cancer cells with radiation or UV. The means of irradiation is not specifically limited. For example, 1-10 Gy of gamma rays can be used. UV rays (wavelength of 100-400 nm, and preferably 290-400 nm) can be delivered employing an appropriate UV irradiation apparatus (manufactured by Funakoshi, Dermaray, Keyence, and the like).

Furthermore, it is possible to efficiently suppress cancers by additionally contacting an anticancer agent with cells (particularly, cancer cells) containing p53 localized in the nuclei. Alternatively, cancers can also be suppressed by embolizing vessels (such as blood vessels or lymph vessels) around the above-mentioned cancer cells that contain p53 localized in nuclei.

The “anticancer agents” include alkylating agents, antimetabolites, antimicrotubule drugs, platinum complex compounds, molecular target therapeutic drugs, and the like. Specific examples of these anticancer agents include, but are not limited to:

<Alkylating agents>
mustards: cyclophosphamide (endoxan), merphalan, and others.
aziridines: thiotepa, and others.
alkylsulfones: busulfan, and others.
nitrosoureas: nimustine, lomustine, and others.
<Antimetabolites>
folic acid derivatives: methotrexate, and others.
purine derivatives: mercaptopurine, azathioprine, and others.
pyrimidine derivatives: 5-fluorouracil, tegafur, carmofur, and others.
<Antimicrotubule drugs>
vinca alkaloids: vincristine, vinblastine, and others.
taxane: paclitaxel, docetaxel, and others.
<Hormone-like drugs>
tamoxifen, estrogen, and others.
<Platinum complex compounds>
cisplatin, carboplatin, and others.
<Molecular target therapeutic drugs>
imatinib, rituximab, gefitinib, and others.

The method for contacting an anticancer agent with cancer cells employs the following procedures: methods of adding the anticancer agent to cells or tissues (cancer cells or cancer tissues) including cells having p53 localized in their nuclei; methods of administering the anticancer agent to a patient or animal with cancer; and the like. In these cases, the amount of the anticancer agent to be used is not specifically limited. When the agent is added, however, the amount is 100 pM-100 μM, and preferably 1 nM-10 μM. When the agent is administered to the body of an animal and, for example, when endoxan is used as the anticancer agent, the amount is 0.1-100 mg/kg/day, and preferably 2-25 mg/kg/day. For anticancer agents other than endoxan, the administered amount or the added amount can be appropriately determined by those skilled in the art.

In order to embolize vessels around cancer cells containing p53 localized in the nuclei, thrombi may be formed in blood vessels around a group of cells or tissues containing the tumor cells having p53 localized in the nuclei. Alternatively, blood vessels or lymph vessels may be embolized with fat, air, or gas.

Moreover, the present invention relates to methods for preventing or treating cancer, comprising the administration of a p53 protein-activating agent of the present invention or a cancer therapeutic agent comprising the p53 protein-activating agent as an active ingredient, into an individual (such as a patient). In the prevention or treatment methods of the present invention, the individual is preferably a human, but the individual is not particularly limited and may be a nonhuman animal.

Generally, the administration to individuals can be carried out by methods known to those skilled in the art, including intra-arterial injection, intravenous injection, and subdermal injection. The dose varies depending on the patient's body weight and age, the administration method, and the like, and an appropriate dose can be suitably selected by those skilled in the art.

Furthermore, the present invention relates to the use of the p53 protein-activating agents of the present invention in the production of cancer therapeutic agents.

Moreover, the present invention provides anti-rheumatic agents, including substances having a function of regulating the autoubiquitination of synoviolin protein, as active ingredients. Preferable examples of the above substance of the present invention include low molecular weight compounds. Thus, a preferred embodiment of the present invention provides anti-rheumatic agents comprising a low molecular weight compound having a function of regulating the autoubiquitination of synoviolin protein, as an active ingredient.

Specifically, the present inventors succeeded in constructing an assay system for evaluating the autoubiquitination reaction of the synoviolin protein, and succeeded in obtaining two low molecular weight compounds represented by the aforementioned formulae (I) (compound X) or (II) (compound Y), each of which have a function of regulating synoviolin protein autoubiquitination, using the assay system.

Accordingly, a preferred embodiment of the present invention provides anti-rheumatic agents including a compound of formulae (I) or (II) as an active ingredient.

The compounds represented by formulae (I) and (II) are as described above.

Moreover, the compounds of formulae (I) or (II) serving as active ingredients in the anti-rheumatic agents of the present invention may be in the form of pharmaceutically acceptable salts of these compounds. In addition to salts of these compounds, hydrates, solvates, and isomers of these compounds are also included. These compounds are also expected to have anti-rheumatic effects.

In the present invention, the “salt” is not specifically limited as long as it forms a pharmaceutically acceptable salt with the compound of the present invention(the compounds of formulae (I) or (II)). Examples of the salt include inorganic salts, organic acid salts, inorganic basic salts, organic basic salts, and acidic or basic amino acids. In general, in the present invention, the “salt” refers to pharmaceutically acceptable salts. Preferred embodiments of the salt are described above.

Moreover, the present inventors discovered that the compounds of formulae (I) and (II) mentioned above have an effect of suppressing the proliferation of rheumatoid arthritis synovial cells (RASCs). Thus, a preferred embodiment of the present invention provides anti-rheumatic agents wherein the compounds of formulae (I) or (II) mentioned above have an effect of suppressing the proliferation of RASCs.

As described above, the anti-rheumatic agents of the present invention have an effect of suppressing the proliferation of RASCs. For example, in chronic rheumatoid arthritis (RA), one of the rheumatic diseases, hyperproliferation and activation of RASCs appears as a principal pathological change. Accordingly, if the suppression of RASC proliferation is possible, this may be useful for treating rheumatic diseases including chronic RA.

The term “rheumatism/rheumatic” in the present invention includes so-called rheumatic diseases. Specific examples include rheumatism, rheumatoid arthritis, acute rheumatoid arthritis, chronic rheumatoid arthritis, malignant rheumatoid arthritis, juvenile rheumatoid arthritis, and the like. Moreover, the rheumatic diseases may be accompanied by other diseases.

The “anti-rheumatic agents” of the present invention can also be referred to as “agents for suppressing the proliferation of RASC”, “agents for suppressing the generation of RASC”, “agents for treating rheumatism”, “agents for treating rheumatic diseases”, “anti-rheumatic drugs”, or “anti-rheumatic disease drugs”.

The “treatment/therapy” in the present invention includes preventive effects, which are capable of suppressing the onset of rheumatic diseases. Moreover, the treatments/therapies are not necessarily limited to complete therapeutic effects on tissues with proliferating RASCs, but may also show partial effects.

The agents of the present invention are as described above. When the agents of the present invention are used, the application site or rheumatic disease to be treated is not particularly limited. For example, they are applied to rheumatism, rheumatoid arthritis, acute rheumatoid arthritis, chronic rheumatoid arthritis, malignant rheumatoid arthritis, juvenile rheumatoid arthritis, and the like. These rheumatic diseases may be accompanied with other diseases.

Moreover, the present invention provides methods, using synoviolin gene expression level as an index, to screen for anti-rheumatic agents. The compounds lowering (suppressing) synoviolin gene expression level are expected to be drugs for rheumatic disease treatment. Using the screening methods for anti-rheumatic agents of the present invention, anti-rheumatic agents or candidate compounds for anti-rheumatic agents can be efficiently obtained.

A preferred embodiment of the screening methods for anti-rheumatic agents of the present invention is a method comprising the following steps (a) to (c):

(a) contacting a test compound with a cell expressing the synoviolin gene;

(b) measuring the expression level or activity of the synoviolin gene in the cell; and

(c) selecting a compound lowering the expression level or activity as compared to when the measurement is carried out in the absence of the test compound.

Each step in the screening method is as described above. The compounds selected by the present screening method serve as anti-rheumatic agents or candidate compounds for anti-rheumatic agents.

Moreover, another embodiment of the screening methods for anti-rheumatic agents of the present invention is a method of selecting a compound lowering the synoviolin gene expression level by using the expression of a reporter gene as an index.

A preferred embodiment of the above screening method of the present invention is a screening method for anti-rheumatic agents comprising the following steps (a) to (c):

(a) contacting a test compound with a cell or cell extract that contains DNA of a structure in which the transcriptional regulatory region of the synoviolin gene and a reporter gene are operably linked to each other;

(b) measuring the expression level of the reporter gene; and

(c) selecting a compound lowering the reporter gene expression level as compared to when the test compound is not contacted.

Each step in the screening method is as described above. The compounds selected by the present screening method serve as anti-rheumatic agents or candidate compounds for the anti-rheumatic agents.

Moreover, the present inventors succeeded in constructing an assay system for evaluating the autoubiquitination reaction of synoviolin. Specifically, as shown in the following Examples, a simple assay system using ELISA was constructed, and the signal showing the synoviolin autoubiquitination reaction was found to be enhanced in the presence of ATP depending on the concentration of anti-HA antibody, secondary antibody, GST-Syno ATM, and the reaction time. Moreover, they discovered for the first time that the low molecular weight compounds of formulae (I) or (II) mentioned above, screened by this assay system, showed an effect of suppressing the proliferation of RASCs, and had anti-rheumatic effects.

Thus, another embodiment of the screening method for anti-rheumatic agents of the present invention is a method using synoviolin protein autoubiquitination as an index. Anti-rheumatic agents can be screened by selecting substances regulating the autoubiquitination of the synoviolin protein of the present invention. For example, the above method of the present invention is a method of screening for anti-rheumatic agents comprising the following steps (a) to (c):

(a) contacting a synoviolin protein and a test compound;

(b) measuring the autoubiquitination of the synoviolin protein; and

(c) selecting a compound regulating the autoubiquitination as compared to when the test compound is not contacted.

In the above method, first, a synoviolin protein and a test compound are contacted. Next, the degree of the autoubiquitination of the synoviolin protein is measured. The degree of the synoviolin protein autoubiquitination can be measured by those skilled in the art using, for example, methods described in the Examples below.

In the above method, next, compounds regulating the degree of the autoubiquitination as compared to when the test compound is not contacted (control) are selected. The compounds regulating the degree of autoubiquitination serve as anti-rheumatic agents or candidate compounds for anti-rheumatic agents.

In the present invention, the term “regulate” includes upregulation (such as promotion) and downregulation (such as suppression).

Moreover, the present invention provides kits comprising various agents, reagents and the like to be used for performing the methods of screening for anti-rheumatic agents of the present invention.

For the kits of the present invention, for example, appropriate reagents can be selected from amongst the various abovementioned reagents of the present invention, depending on the screening method to be performed. For example, kits for screening for anti-rheumatic agents of the present invention may include at least a synoviolin protein of the present invention as a component. The kits of the present invention may further include various reagents, containers, and the like, to be used for the methods of the present invention. For example, they may appropriately include anti-synoviolin antibodies, probes, various reaction regents, cells, culture media, control samples, buffers, instructions describing how to use the kits, and the like.

Moreover, the present invention relates to methods for preventing or treating rheumatic diseases, comprising administering to an individual (such as a patient) an anti-rheumatic agent including a substance having a function of regulating the autoubiquitination of the synoviolin protein of the present invention as an active ingredient. In the preventive or treatment method of the present invention, the individual is preferably a human, but is not particularly limited and may be a non-human animal. The administration to an individual is as described above.

Furthermore, the present invention relates to the use of substances having a function of regulating the autoubiquitination of the synoviolin protein of the present invention in the production of anti-rheumatic agents.

All prior-art documents cited herein are incorporated herein by reference.

### EXAMPLES

Herein below, the present invention is further specifically described using Examples, but the invention is not to be construed as being limited thereto.

### Example 1

### Examination of p53 Activation in Cultured MEF Cells

p53 in synoviolin homozygous knockout mouse (syno^{-/-}) MEFs was confirmed by immunofluorescence staining.

In this immunostaining method, MEFs were immobilized onto a slide glass according to a customary method and anti-p53 antibody (mouse monoclonal antibody BD from Becton, Dickinson) was used for immunostaining. The preparation was blocked with 3% bovine serum albumin (BSA) for 30 min, and was then subjected to immunoreaction with 0.3% BSA-diluted anti-p53 antibodies (BD: 10 μg/mL) at room temperature for 60 min. The preparation after reaction was washed with PBS, and then was subjected to immunoreaction with a TRITC-labeled anti-mouse IgG antibody (Dako) as the secondary antibody. The antigen that immunoreacted with the anti-p53 antibodies was examined by fluorescence microscopy.

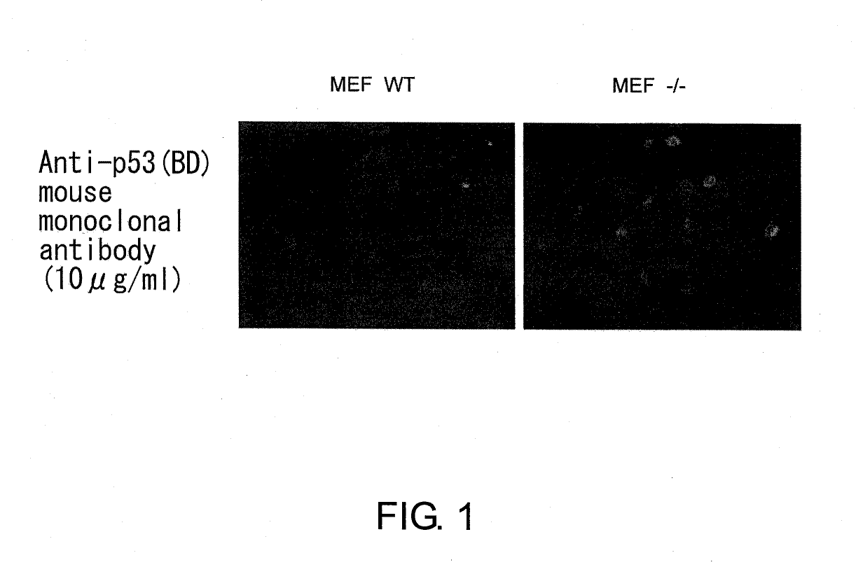

The results demonstrated that a large number of cells showing increased p53 expression and p53 translocation into the nucleus were found in cultured syno^{-/-} MEF cells, as compared to wild-type MEF cells (panel designated “MEF −/−” in FIG. 1).

### Example 2

### Examination of p53 Activation in Syno^{-/-} Mice

The p53 activation in syno^{-/-} mice was examined by immunostaining of embryos.

For immunostaining of syno^{-/-} embryos, the tissue was immobilized onto a glass slide according to a customary method and the Vectastain ABC kit (Vector Laboratories) was used for immunostaining. The preparation was blocked with a blocking reagent for 30 min and then was subjected to immunoreaction with anti-p53 antibody FL393, diluted to 5 μg/mL, at room temperature for 60 min. The preparation after reaction was washed with PBS, and was then subjected to immunoreaction with HRP-labeled anti-rabbit IgG antibody as a secondary antibody. The antigen that immunoreacted with the anti-p53 antibody was detected by color development from added 3,3′-diaminobenzidine tetrahydrochloride, resulting from HRP activity. Methyl green counterstaining was also carried out.

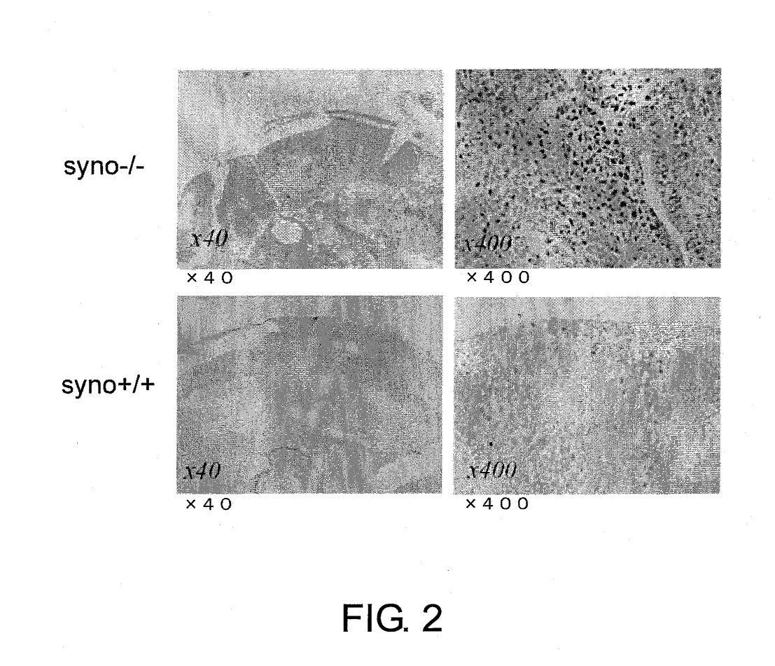

The results confirmed that p53 was stable, and showed increased expression in syno^{-/-} embryos (FIG. 2).

### Example 3

### Effect of Synoviolin on p53

Western blotting was performed to detect p53 in cultured syno^{-/-} MEF cells.

Fractions of homogenized cells were prepared from various cells using a cell homogenizing solution (50 mM Tris-HCl [pH 8.0], 150 mM NaCl, 1% NP40, 1 mM PMSF, 0.1% sodium dodecyl sulfate (SDS), 2 μg/mL leupeptin, 2 μg/mL aprotinin, and 2 μg/mL pepstatin). Then, fractions of homogenized cells were separated by SDS-PAGE. After SDS-PAGE, the cell-derived proteins were transferred onto a nitrocellulose (NC) membrane by an electroblotting method. This NC membrane was blocked with Tris buffered saline (TBS) with 5% skim milk at room temperature for 1 h, and then immunoreacted with anti-p53 antibody C-terminal aa; 195-393 or FL393, both of which were diluted in TBS with 5% skim milk, at room temperature for 1 h. The NC membrane after reaction was washed with 0.1% Tween 20/TBS, and then immunoreacted with horse radish peroxidase (HRP)-labeled anti-rabbit IgG antibody as secondary antibody, at room temperature for 1 h. The membrane was washed with 0.1% Tween 20/TBS, and HRP activity was sought to detect the target antigen. An ECL kit (Amersham) was used to detect HRP activity Clinical Chemistry. 25, p1531, 1979).

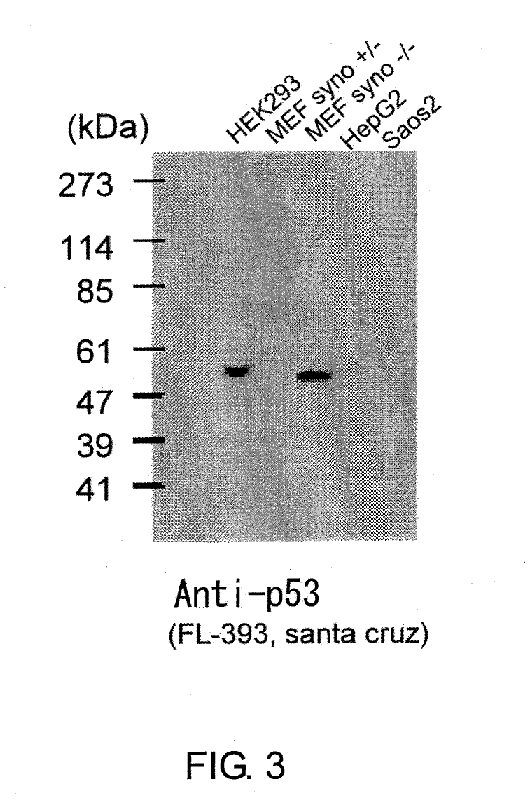

The results of Western blotting confirmed that p53 expression level was increased in the cultured syno^{-/-} MEF cells (FIG. 3).

### Example 4

### Identification of the p53 Phosphorylation Site in Cultured Syno^{-/-} MEF Cells

In the present Example, the phosphorylation site of p53 was identified by Western blotting using anti-p53 antibodies.

Using four types of anti-phosphorylated p53 monoclonal antibodies, which recognize phosphorylations at different serine residues of p53 (SEQ ID NO: 17) (Phospho-p53 [ser15], Phospho-p53 [ser20], Phospho-p53 [ser37], and Phospho-p53 [ser46]; Cell Signaling Technology), proteins of MEF cells were separated by SDS-PAGE, and Western blotting was performed. Western blotting was performed as described in Example 3, except that anti-phosphorylated p53 monoclonal antibodies were used as primary antibodies and anti-mouse IgG sheep-HRP was used as labeled antibody.

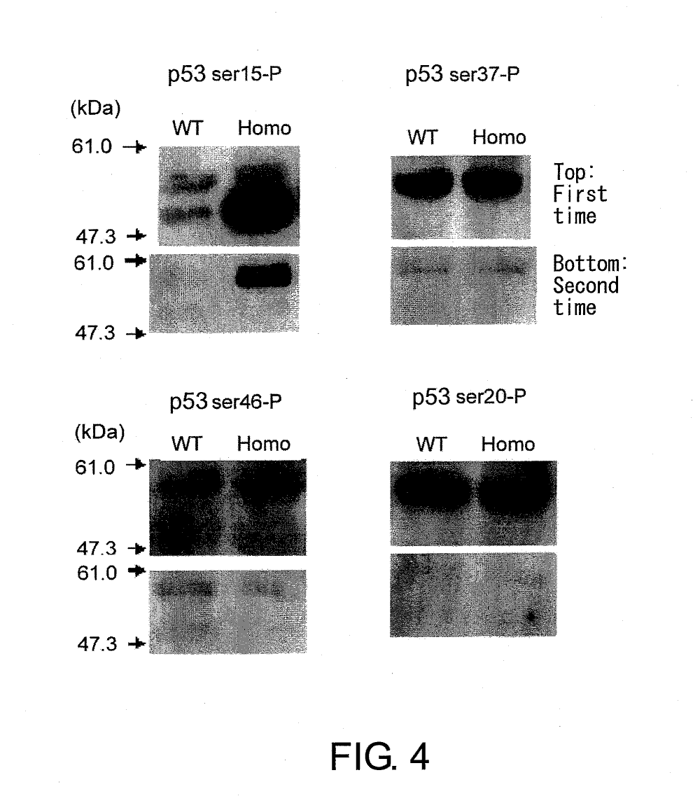

The results showed a remarkable phosphorylation of the 15th serine residue of the p53 amino acid sequence (SEQ ID NO: 17) in the cultured syno^{-/-} MEF cells. In FIG. 4, the top left panel shows the phosphorylation of the 15th serine residue. The band near 53 kDa is remarkably dark.

### Example 5

### Elucidation of the Mechanism for Enhancing the Phosphorylation of Ser15

RKO cells (human colon cancer cells), which had been confirmed to express wild-type p53, were seeded on a 60 mm plate at 1.0×10⁵ cells/plate/2 mL), and were transfected with siRNA oligonucleotides against GFP and synoviolin, using Oligofectamine. After 72 h, caffeine (10 mM), an inhibitor of ATM (ataxia-telangiectasia mutated) and ATR (an ATM and Rad3-related), which are important in the phosphorylation of Ser15, was added. Then, Western blotting was performed using Phospho-p53 (ser15), an antibody to the phosphorylated Ser15-p53.

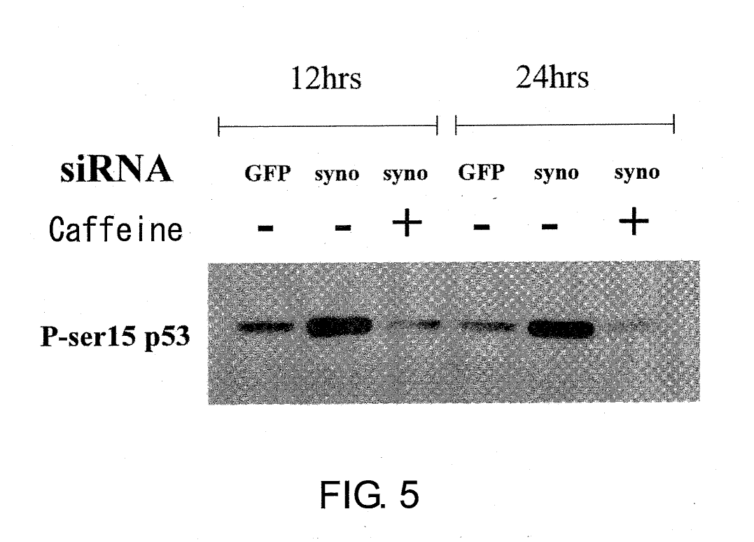

The results showed that the phosphorylation of Ser15 that had been enhanced by siRNA against synoviolin was completely inhibited by the addition of caffeine (both 12 h and 24 h after the addition) (FIG. 5). This suggests that the inhibition of synoviolin expression induces the activation of p53 caused by ATM and ATR.

### Example 6

### Influences of Synoviolin on p21 Expression Induced by p53

RKO cells were treated with siRNA for synoviolin, and changes in the expression of p21, which is a transcription product of p53, were examined by Western blotting.

RKO cells, which had been confirmed to express wild-type p53, were seeded on a 60 mm plate at 1.0×10⁵ cells/plate/2 mL, and were transfected with siRNA oligonucleotides against GFP and synoviolin using Oligofectamine. After 72 h, the cells were collected and prepared. The prepared proteins were separated by SDS-PAGE, and Western blotting was performed using anti-p21 polyclonal antibodies (Santa Cruz Biochemicals). Western blotting was performed as described in Example 3, except that anti-p21 polyclonal antibodies were used as the primary antibody and anti-mouse IgG sheep-HRP was used as secondary antibody.

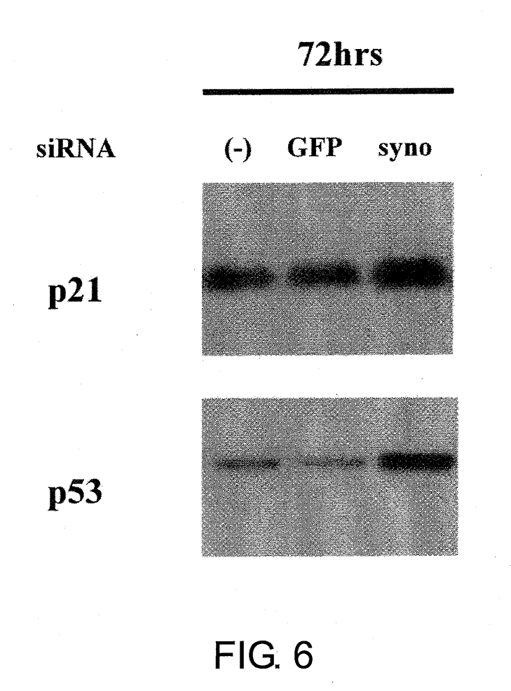

The results showed that treatment with siRNA for synoviolin increased p53 expression and p21 expression concurrently. This effect was clearly shown at 72 h (FIG. 6).

### Example 7

### Examination of Inhibition of Synoviolin Expression on the Cell Cycle

In the present Example, effects of the inhibition of synoviolin expression on the cell cycle, in rheumatoid patient-derived synovial cells, using the siRNA effect, were examined.

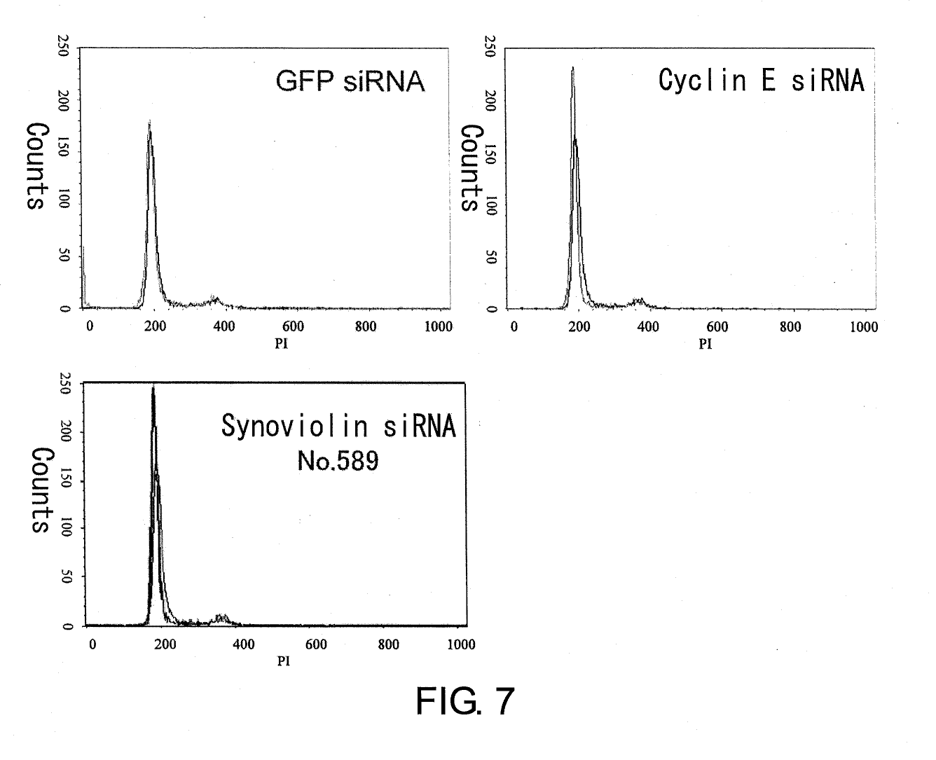

RA synovial cells were seeded on a 10 cm dish at 9.0×10⁴ cells/dish, and were transfected with synoviolin siRNA (final concentration 25 nM). Then, the cell cycle was monitored by flow cytometer. The results showed that a delay in the cell cycle at the G0/G1 phase was observed after treatment with 25 nM of siRNA (FIG. 7).

The siRNA used is h589.

h589 is a double-stranded RNA in which the following sense and antisense strands are annealed:

| | |
|---|---|
| Sense strand h589: | |
| GGU GUU CUU UGG GCA ACU G TT | (SEQ ID NO: 18) |
| Antisense strand h589: | |
| CAG UUG CCC AAA GAA CAC C TT | (SEQ ID NO: 19) |

### Example 8

### Examination of Synoviolin Expression in Tumor Tissues

A tissue array (Chemicon; 10 common human cancer tissues with normal human tissues) was immunostained using anti-synoviolin antibodies (10 Da). For immunostaining, the concentration of the antibodies was 8 μg/mL, and the kit used is Simple Stain MAX (M).

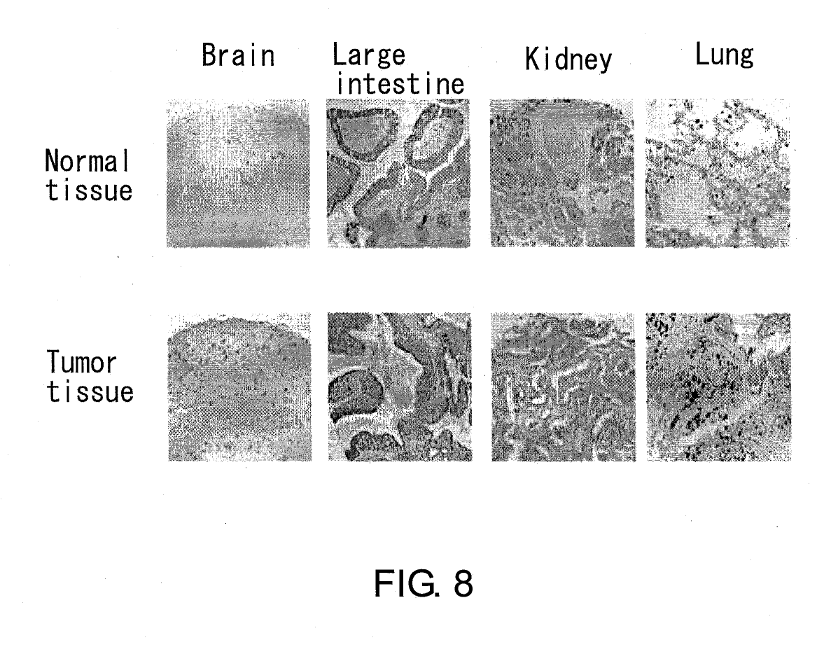

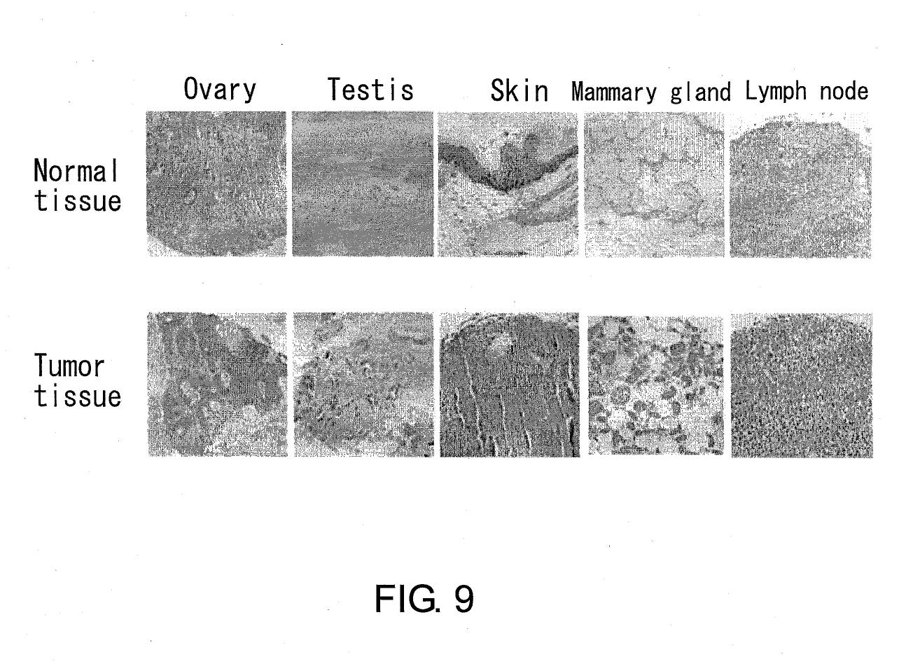

The results showed that, in normal tissues, synoviolin expression was found in the large intestine, the kidney, the lung, the ovary, the testis, the skin, and the mammary gland, but not in neural tissue or lymph nodes. Moreover, synoviolin expression was also seen in the corresponding tumor tissues. In particular, tissues with apparently enhanced synoviolin expression were neural tissue and lymph nodes (FIG. 8 and FIG. 9).

### Example 9

### Influences of the Coexpression of Synoviolin and p53, in Cultured Cells, on the Localizations of These Proteins

Three types of plasmids, GFP-p53, FLAG-synoviolin, and FLAG-synoviolin C307S (without ubiquitination (Ub) activity), were introduced into Saos-2 cells.

The respective plasmids were as follows:

GFP-p53: expresses a fusion protein composed of green fluorescence protein and wild-type p53
FLAG-synoviolin: expresses wild-type synoviolin with a FLAG tag
FLAG-synoviolin C307S (without ubiquitination (Ub) activity): expresses deactivated synoviolin with a FLAG tag.

Transfection was performed using FuGENE6 (Roche). After 24 h, the cells were fixed with 10% formalin and the nuclei were stained with 400-fold diluted primary α-FLAG antibody, 200-fold diluted secondary antibody α-mouse IgG-TRITC, and 1 μM DAPI, to observe protein localizations.

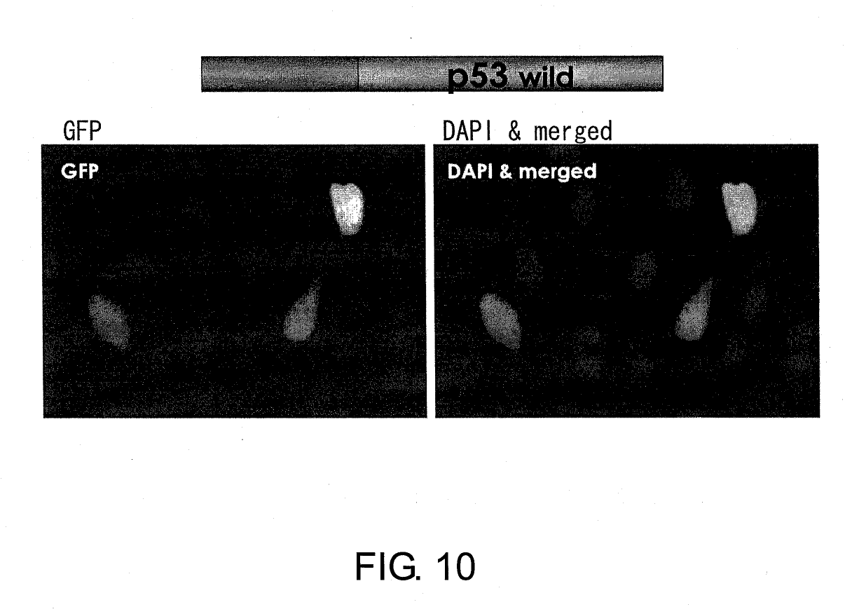

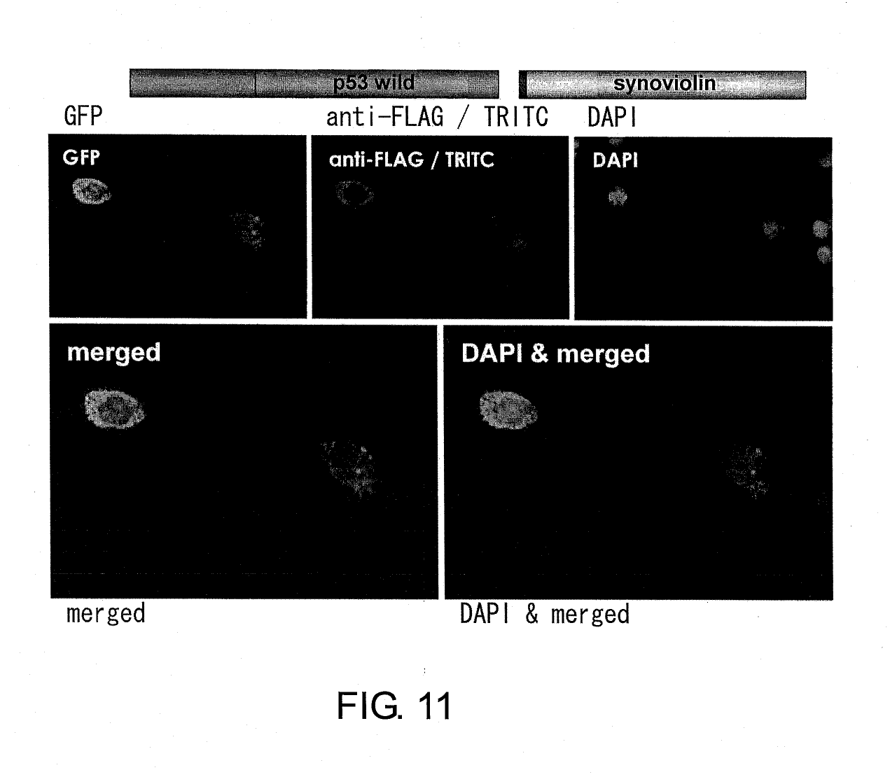

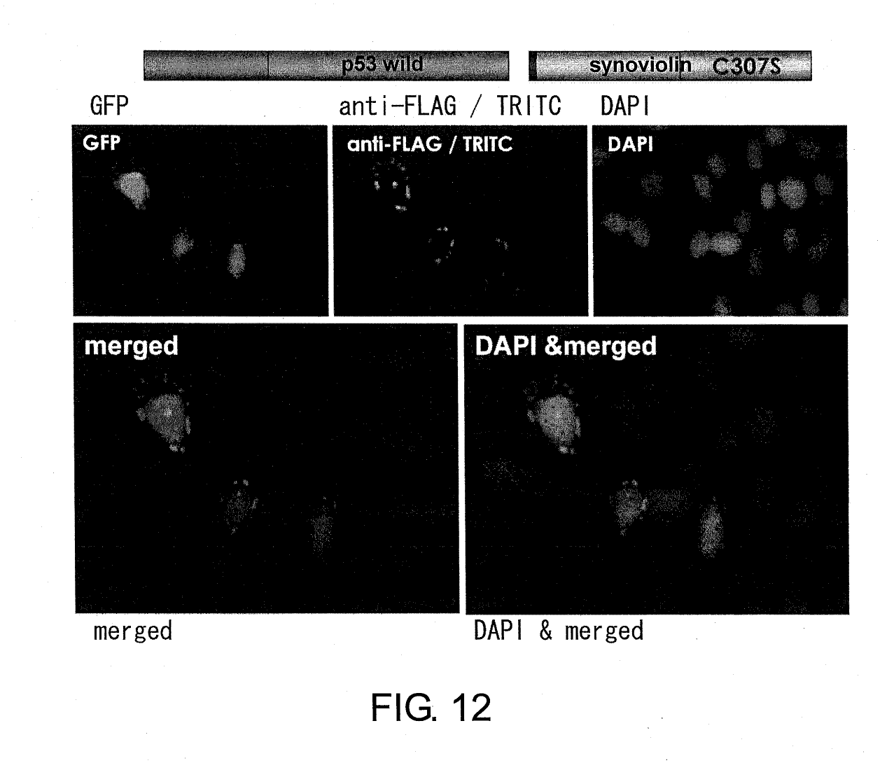

The results showed that wild-type p53 localized to the nucleus when overexpressed (FIG. 10). Wild-type synoviolin localized to the cytoplasm (in particular, to the region around the nucleus) when overexpressed. Moreover, when wild-type p53 and wild-type synoviolin were overexpressed, it was observed that p53, which normally localizes in the nucleus, was distributed around the nucleus in a punctuate manner, and colocalized with synoviolin(FIG. 11). When wild-type p53 and the synoviolin C307S mutant protein were overexpressed, it was observed that p53 formed a large dot in the cytoplasm, and colocalized with synoviolin (FIG. 12).

These tests showed that synoviolin and p53 colocalized under certain conditions. The nature of the colocalization may change depending on the presence or absence of the ubiquitination activity.

### Example 10

### Examination of GST-p53 in Vitro Ubiquitination by MBP-Synoviolin ΔTM-His

It has been observed that the p53 protein level in the cell changed with alterations in synoviolin levels within a cell, suggesting that synoviolin controls p53. Therefore, in order to investigate whether synoviolin directly ubiquitinates p53, an in vitro ubiquitination reaction was examined using GST-p53 and MBP-Synoviolin ΔTM-His.

GST-p53: a fraction obtained by purifying p53 fused N-terminally with GST and expressed in E. coli.
MBP-Synoviolin ΔTM-His: a fraction obtained by purifying synoviolin fused N-terminally with MBP and C-terminally with His tag and expressed in E. coli.

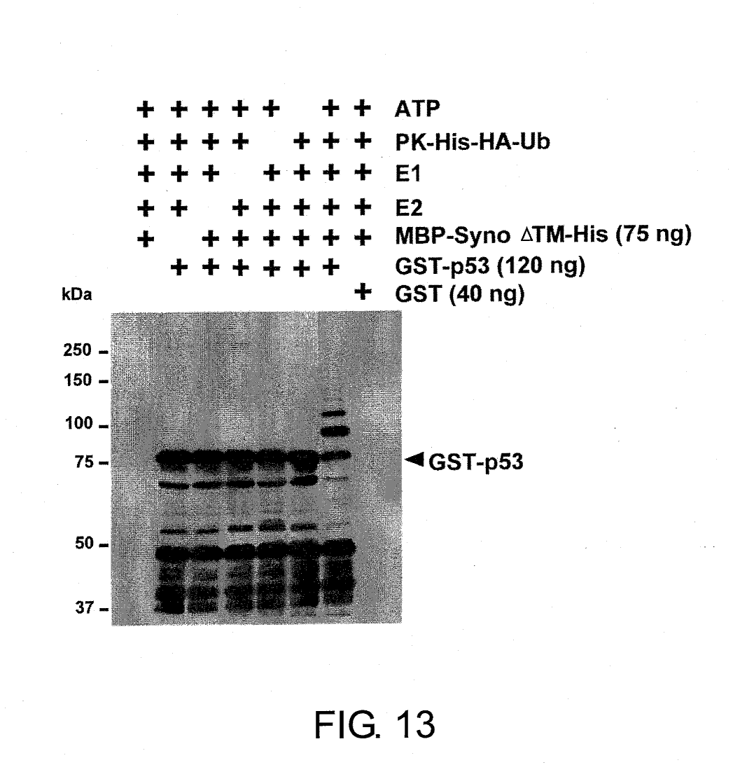

E. coli (BL21) hosting pGEX/p53 was cultured in 500 mL of LB medium. After induction with IPTG (1 mM at 30° C. for 6 h), an extract was prepared from the culture solution, using buffer containing 0.5% NP-40. From the E. coli extract, GST-p53 was purified using a GSH-Sepharose resin in the presence of 0.1% NP-40. The dialyzed sample was used for reaction with the combination of MBP-Synoviolin ΔTM-His and other compositions used in the in vitro ubiquitination reaction (ATP, PK-His-HA-Ub, yeast E1, and His-UbCH5c) (FIG. 13). After the reaction, proteins were separated by 7.5% SDS-PAGE, and transferred onto a PVDF membrane, prior to detection of the p53 protein on the membrane using anti-p53 antibodies (FL393 or DO-1). Reaction and detection were performed with variations in the amount of GST-p53 added.

The results showed that p53-derived ladder-shaped signals were observed, centering around 90 kDa, when all compositions, including GST-p53 and MBP-Synoviolin ΔTM-His, were added (FIG. 13). From these results, synoviolin can be said to be directly involved in p53 ubiquitination. Accordingly, it was shown that p53 ubiquitination can be suppressed by inhibiting the expression and/or function of synoviolin.

### Example 11

### Examination of the mRNA Levels of Synoviolin and p53 Under RNAi

In this Example, changes in the mRNA levels of synoviolin and related genes were examined over time under conditions of synoviolin RNAi, to examine the influence of synoviolin on the cell cycle, apoptosis, and the like.

RASCs were seeded on a 10 cm dish at 30,000 cells/dish, and were transfected with 25 nM of siRNA (No. 589) according to a routine method. The cells were then cultured for 4 d. During this period, cells were collected overtime to obtain mRNA. Random primers were used, with 1 μg of mRNA as a template, to perform reverse transcription reactions, and cDNAs were obtained. The obtained cDNAs were quantified using the ABI TaqMan Gene expression assay (GEX). The mRNA levels were calculated using 18S rRNA as control.

The GEX reagent target assay no. (Assay ID) is Hs00381211_m1 for synoviolin and Hs00153340_m1 for p53.

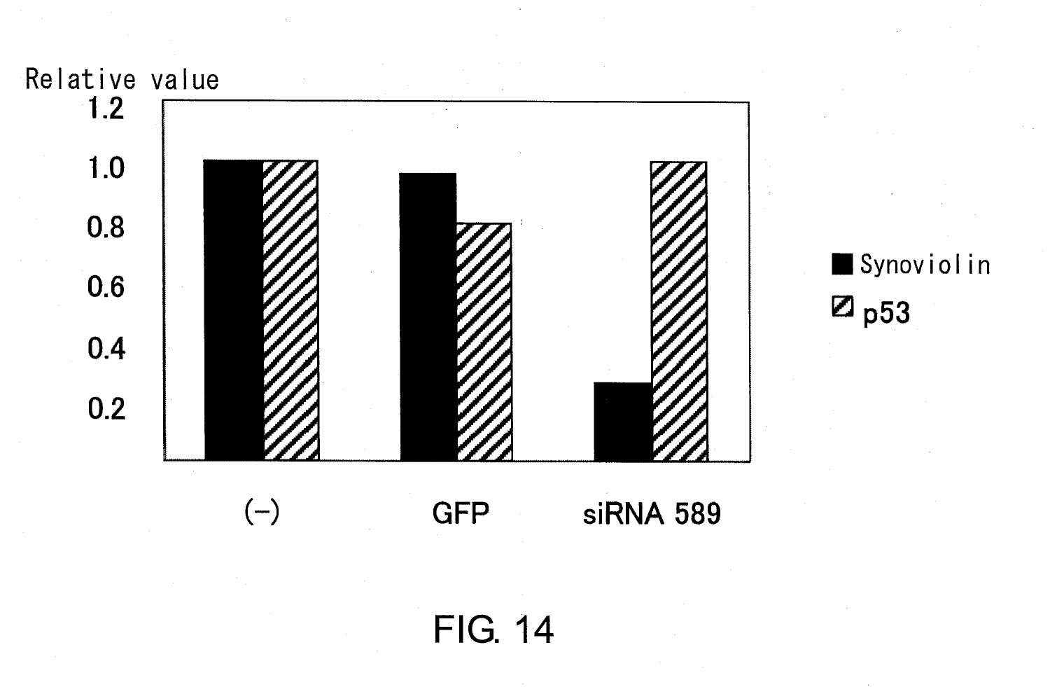

The results confirmed that the level of p53 mRNA was unchanged whereas the level of synoviolin mRNA decreased in the presence of synoviolin siRNA (FIG. 14).

### Example 12

### Determination of the p53-Binding Domain in Synoviolin

GST-synoviolin bound to p53 in the in vitro pulldown assay. For this binding, the 35 amino acid residues from positions 236-270 in the amino acid sequence of the synoviolin protein (SEQ ID NO: 2) were both necessary and sufficient.

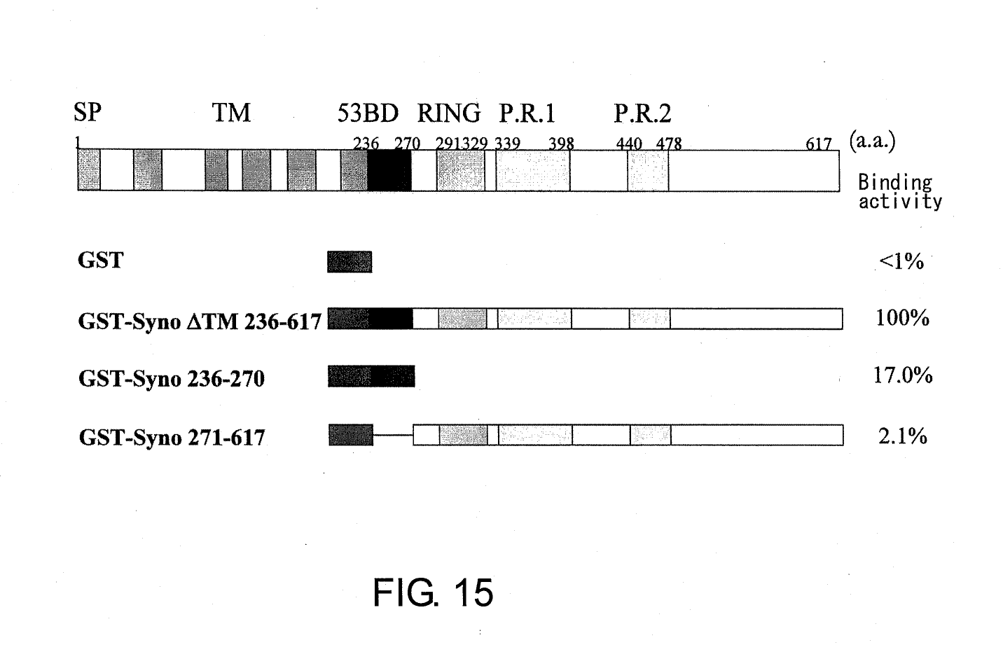

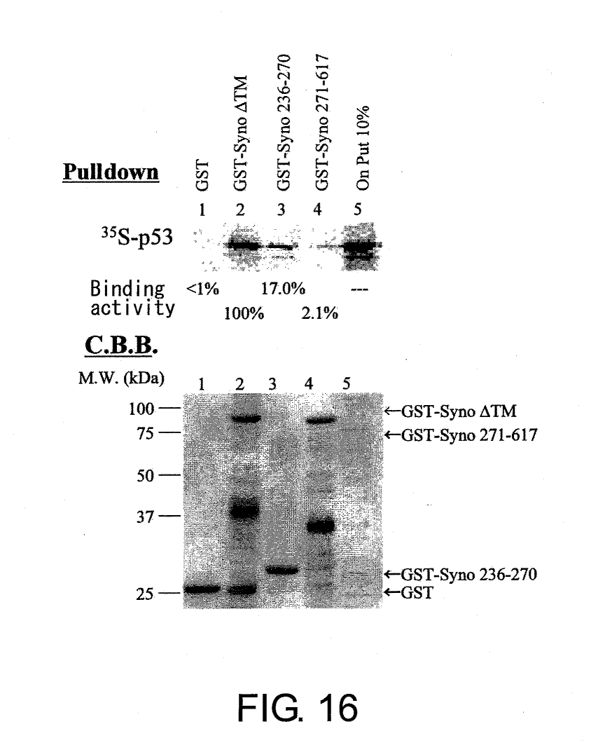

To identify a p53 domain necessary for synoviolin to bind to p53, the present Example produced synoviolin binding domain deletion variants as shown in FIG. 15 and carried out a GST pulldown assay using [³⁵S]-p53 (FIG. 16).

One hundred μL of competent cells (BL-21 strain) were transfected with 1 μL of plasmids encoding various GST proteins. The name of each GST protein and plasmid was as follows:

GST Protein:Plasmid

GST:pGEX-6P-1 (Pharmacia Biotech)

GST-SynoΔTM 236-617:pGEX-5-1/S ΔTM

GST-SynoΔTM 236-270:p6-3

GST-SynoΔTM 271-617:pST490

The cells were inoculated into 4 mL of LB-Amp⁺ and cultured at 37° C. overnight. On the next day, the OD₆₀₀ values of the pre-cultures were measured, and cultures at OD₆₀₀ values of 3.0 were inoculated into 15 mL of LB-Amp⁺ (final concentrations 0.2). Cultures were grown in a thermostat bath at 25° C. for about 2 h. After confirming that the OD₆₀₀ values had reached 0.6-0.8, ice was added to the bath to cool it to 20° C. The culture container was held therein for 10 min to cool to 20° C. Fifteen μL of 0.1 M IPTG (final concentration 0.1 mM, 1/1,000 the usual concentration) and 150 μL of 1 mM ZnCl₂ (final concentration 10 μM) were added, and shaking culture was then performed at 20° C. for 4 h to induce the expression of GST proteins. After induction, cells were collected by centrifugation (5000 rpm for 5 min at 4° C.). Cells were resuspended in 1 mL of PBS(−), transferred to an Eppendorf tube, and collected (at 14,000 rpm for 1 min at 4° C.). After completely aspirating the supernatant, the cells were resuspended in 500 μL of PBS(−)/Z (PBS(−) with 10 μM ZnCl₂), followed by freezing in liquid nitrogen and preservation at −20° C. On the next day, the samples at −20° C. were thawed by placing in a thermostat bath at 37° C. for 10 min, and were then placed in ice water to cool to 0° C. The following protease inhibitors were mixed, and 6.5 μL of the mixture was added to each sample.

| | | |
|---|---|---|
| 100 mM PMSF (Final 1 mM) | 20 | μL |
| Aprotinin (Final 0.1%) | 2 | μL |
| 0.5 mg/mL Pepstatin A (Final 0.5 μg/mL) | 2 | μL |
| 1 mg/mL Leupeptin (Final 1 μg/mL) | 2 | μL |

Each sample was subjected to ultrasonic disruption (power level 7; 15 sec, three times). Each sample was placed in ice water for 30 sec to cool after each burst. Next, 500 μL of 2×GST buffer/Z (2% Triton X-100, 720 mM NaCl, 1×PBS(−), 10 μM ZnCl₂, 10 mM β-mercaptoethanol, 2 mM PMSF, and 0.1% aprotinin) was added and mixed, followed by further ultrasonic disruption (power level 7; 15 sec, once). The disrupted solution was centrifuged at 14,000 rpm for 30 min at 4° C. During this period, 200 μL of an 80% slurry of Glutathione Sepharose beads was washed with 1 mL of 1×PBS(−) three times, and 160 μL of 1×PBS(−) was added thereto to adjust the slurry to 50%. Eighty μL of this 50% slurry of Glutathione Sepharose beads was added to 1 mL of a supernatant after centrifugation, and the mixture was rotated at 4° C. for 2 h, to bind GST proteins to the beads. The beads were washed four times with 1 mL of 1×GST-buffer/Z (1% Triton X-100, 360 mM NaCl, 0.5×PBS(−), 5 μM ZnCl₂, 5 mM β-mercaptoethanol, 1 mM PMSF, and 0.05% aprotinin). Centrifugation was performed at 2,000 rpm for 1 min at 4° C. The supernatant was completely aspirated. Then, the pellet received 60 μL of 1×GST-buffer/Z to make the total volume to 100 μL. Ten μL thereof was mixed with the same volume of 2×SDS sample buffer, heated in a heat block at 100° C. for 5 min, and applied to a 10% gel. At the same time, 0.25-4 g of BSA was applied. Following electrophoresis (150 V, 50 min), CBB staining (with fresh CBB, 30 min), decolorization (1 hr, twice), glycerol water treatment (30-60 min), and gel drying (80° C., 1 h), the expression levels and recovery efficiencies of GST proteins were checked. On the next day, [³⁵S]-p53 was translated in vitro. First, the following reagents were mixed:

| | | |
|---|---|---|
| TNT reticulocyte lysate | 25 | μL |
| TNT reticulocyte buffer | 2 | μL |
| Amino acid mixture (-Met) | 1 | μL |
| DEPC-treated water | 15 | μL |
| RNase inhibitor | 1 | μL |
| TNT polymerase | 1 | μL |
| [³⁵S]-Met | 4 | μL |
| Plasmid (p53-HA) | 1 | μL |
| Total | 50 | μL |

In vitro translation was performed in a thermostat bath at 30° C. for 1.5-2.5 h. Meanwhile, the lid of a G-25 column was loosened, and mild centrifugation (at 2,500 rpm for 1 min at 4° C.) was performed. Then, 100 μL of pulldown buffer V (20 mM HEPES pH 7.9, 150 mM NaCl, 0.2% Triton X-100) was placed thereon, and further centrifugation was performed. The column was then washed. A total of 50 μL of the in vitro translation solution was placed in this column, and centrifugation (at 2,500 rpm for 1 min at 4° C.) was performed. A further 200 μL of pulldown buffer V was placed on the column, and centrifugation (at 2,500 rpm for 1 min at 4° C.) was performed once more. This material was used as the in vitro translation product (IvTL). Four μL of this product was mixed with 16 μL of Milli-Q water and 20 μL of 2×SDS buffer, to prepare the substrate termed “On put 10%”. One hundred and twenty μL of IvTL was added to 1 mL of pulldown buffer V containing beads bonded with 30 μg of GST protein, and then rotated at 4° C. for 1 h. Following centrifugation (at 10,000 rpm for 1 min at 4° C.), 370 μL of supernatant was added to each 1 mL of pulldown buffer V containing GST or GST-synoviolin beads, and then rotated at 4° C. for 1 h. These beads were washed four times with 1 mL of pulldown buffer V. During washings, about 100 μL of the supernatant was always left in the tubes, so as not to aspirate the beads. Centrifugation was performed at 2,500 rpm for 1 min at 4° C. After the supernatant was aspirated, 40 μL of 1×SDS sample buffer was added to the tube for use as the pulldown sample. The “On put 10%” and the pulldown sample were heated at 100° C. for 5 min, and then held at −20° C. On the next day, the samples were warmed in a thermostat bath at 37° C. for 10 min, and 10 μL of each sample was applied to a 10% gel. Following electrophoresis (150 V, 50 min), CBB staining (30 min), decolorization (1 h×2), glycerol water treatment (30-60 min), and gel drying (80° C., 1 h), the gel was exposed to an IP plate. After 14 h, the exposed IP plate was read by BAS, and quantified by ImageGauge software. Moreover, the CBB-stained gel was read by a film scanner.

The results showed that p53-binding domain deletion variants almost completely lost binding activity. Moreover, from FIG. 15, the p53-binding domain is considered to be a single region of 35 amino acids from amino acid 236-270.

### Example 13

### Influences of Compounds X and Y on the p53 Ubiquitination Activity of Synoviolin

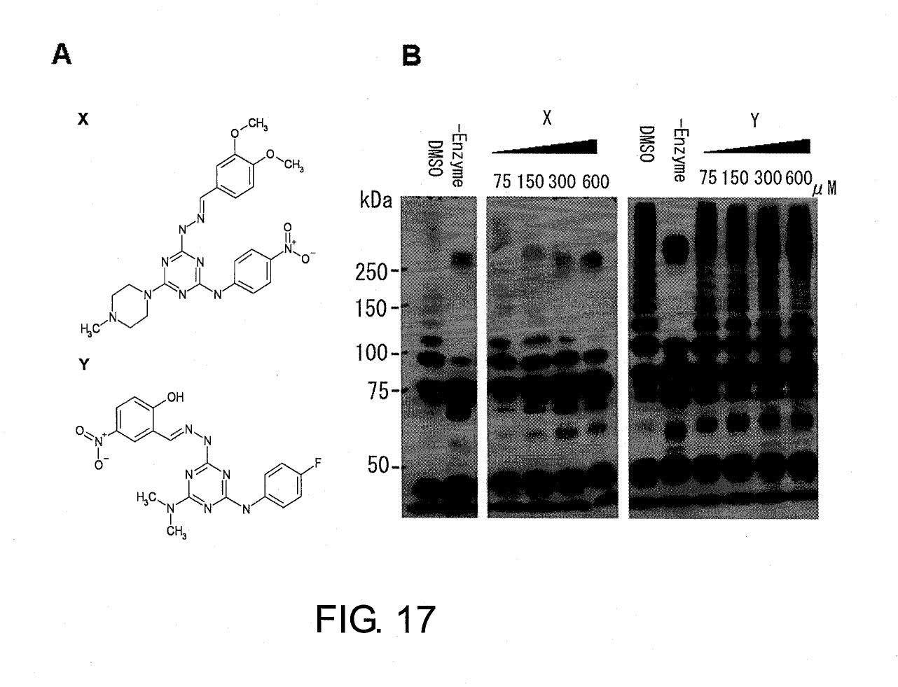

In order to examine the in vitro ubiquitination reaction of GST-p53 by synoviolin, the influences of compounds X and Y on this reaction were examined. The chemical formulae of compounds X and Y are shown in FIG. 17A.

In the presence of GST-p53, varying concentrations (75 μM, 150 μM, 300 μM, or 600 μM) of compounds X and Y were added to the in vitro ubiquitination reaction system using MBP-Syno ΔTM-His. After reaction, proteins were separated by 7.5% SDS-PAGE, transferred onto a PVDF membrane, and then detected using the anti-p53 antibody FL393 (Santa Cruz Biotechnology).

The results showed that compounds X and Y inhibited the GST-p53 ubiquitination reaction in a concentration-dependent manner (FIG. 17B).

### Example 14

### Examination of Cytotoxicity of Compounds X and Y

RKO cells (a human colon carcinoma cell line; ATCC: CRL-2577) were seeded in Eagle's Minimum Essential Medium containing 10% fetal bovine serum in a 96-well microtiter plate (Falcon; non-coated) at 1,000 cells/100 μL/well, and cultured for 24 h. Compounds X and Y were dissolved in Eagle's Minimum Essential Medium containing 10% fetal bovine serum, and 10 μL amounts were added to wells. The cells were then cultured for 24-72 h.

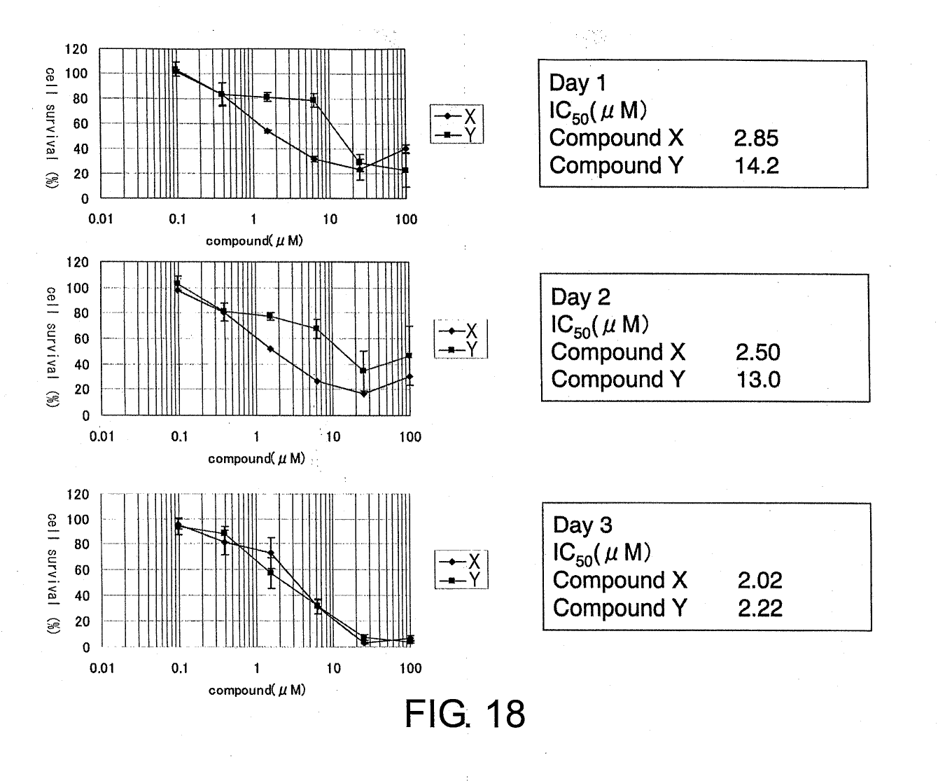

After culture, WST-8 (2-(2-methoxy-4-nitrophenyl)-3-(4-nitrophenyl)-5-(2,4-disulfophenyl)-2H-tetrazolium, monosodium salt) was added to each well. After 2-4 h, absorbances at 450 nm were measured (reference wavelength: 750 μm) and cell survival rates were calculated (FIG. 18).

The calculation method was based on the following formula:

$$Cell survival rate ( \% ) = \frac{( Specimen ) - ( Blank )}{( Negative control ) - ( Blank )} \times 100$$

The results showed that cell proliferation was suppressed by compounds X and Y (FIG. 18).

### Example 15

### Influences of Compounds X and Y on p53 Protein Expression Level in Cultured Cells

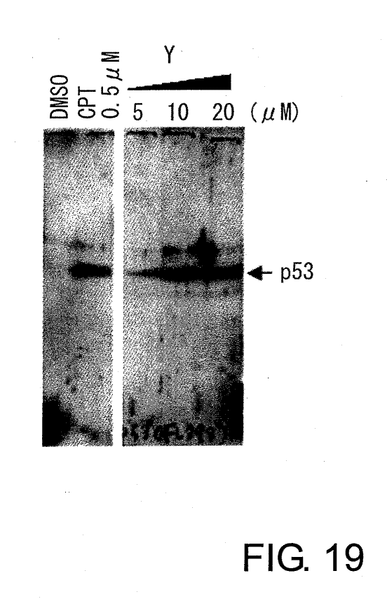

RKO cells were treated with compound Y, and changes in p53 protein expression level were examined by Western blotting. Specifically, RKO cells were seeded in 10 cm plates at 5.0×10⁵ cells/plate, and cultured for 24 h. Then, compound Y was added at final concentrations of 5 μM, 10 μM, or 20 μM. After 24 h the cells were collected and cell lysates were prepared. Proteins were separated by SDS-PAGE and subjected to Western blotting using the anti-p53 antibody FL393 (Santa Cruz Biotechnology Inc.). The Western blotting procedure was the same as described in Example 3, except that anti-p53 antibody was used as a primary antibody and an anti-human IgG rabbit-HRP was used as secondary antibody. The results showed that p53 protein expression level was increased by compound Y (FIG. 19).

### Example 16

### Construction of an ELISA-Based Evaluation System for the Synoviolin Autoubiquitination (Auto-Ub) Reaction

The aim was to construct a simple, ELISA-based, assay system to measure the auto-Ub reaction, and evaluate compounds that inhibit the auto-Ub reaction.

First, the concentration of antibodies in the ELISA-based detection system for the synoviolin auto-Ub reaction was examined.

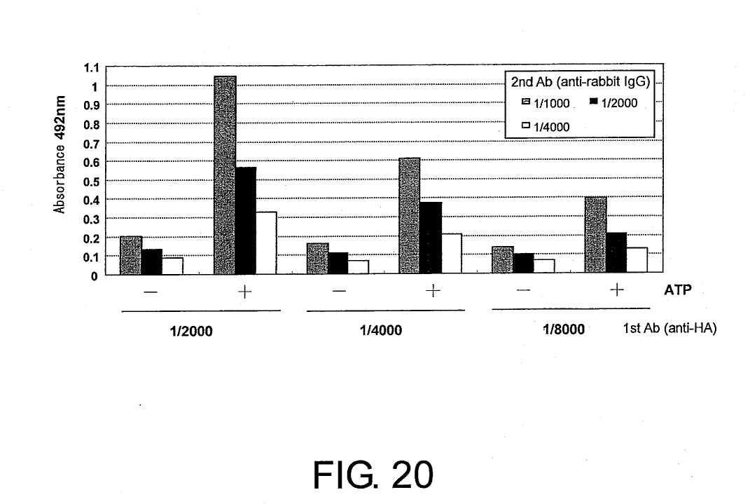

Specifically, His-E1 (human, 125 ng), His-PK-HA-Ub (1.6 μg), His-UBE2G2 (0.8 μg), and GST-Syno ΔTM (0.4 μg) were mixed in the presence or absence of ATP (10 mM), and the in vitro ubiquitination reaction (30 μl) was performed at 37° C. for 1 h. After adding 70 μl of 0.5M EDTA to the reaction solution to terminate the reaction, the mixed solution was added to a glutathione-coated plate (Reacti-Bind; Pierce) for ELISA, that had been previously blocked with PBST/10% skim milk for 2 h. The plate was shaken at room temperature for 1 h. After the liquid was discarded, the plate was then washed three times with 200 μl of PBST. One hundred μl amounts of anti-HA antibody diluted with PBST ( 1/2,000, 1/4,000, and 1/8,000) were dispensed, and the plate shaken at room temperature for 1 h. After the liquid was discarded, the plate was washed three times with 200 μl of PBST. One hundred μl amounts of anti-rabbit IgG antibody diluted with PBST ( 1/1,000, 1/2,000, and 1/4,000) were dispensed, and the plate shaken at room temperature for 30 min. After the liquid was discarded, the plate was washed three times with 200 μl of PBST. One hundred pt of an OPD color developing solution was added and the plate shaken at room temperature for 15 min. Then, 50 μL of 2 N sulfuric acid was added to terminate the reaction, and the absorbances at 492 nm measured using a plate reader (FIG. 20).

Next, the concentration of GST-Syno ATM in the ELISA-based detection system for the synoviolin auto-Ub reaction was examined.

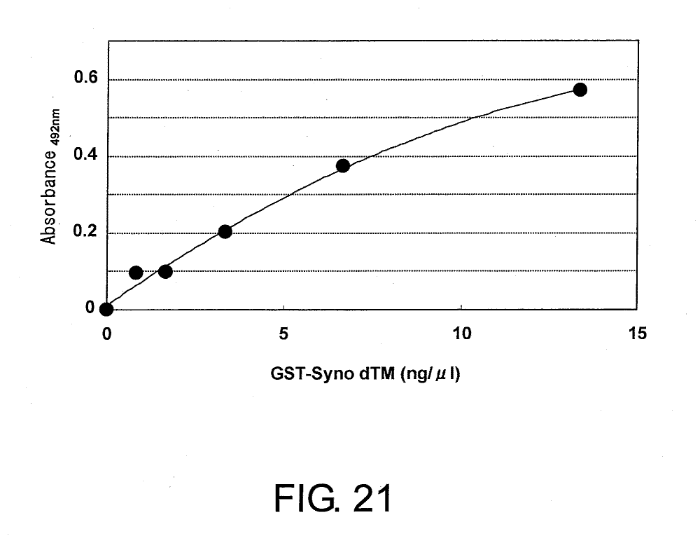

Specifically, ATP (10 mM), His-E1 (human, 125 ng), His-PK-HA-Ub (1.6 μg), His-UBE2G2 (0.8 μg), and GST-Syno ATM (0.83, 1.7, 3.3, 6.7, or 13.3 ng/μL) were mixed, and the in vitro ubiquitination reaction (30 μL) was performed at 37° C. for 1 h. After adding 70 μl of 0.5M EDTA to the reaction solution to terminate the reaction, the mixed solution was added to a glutathione coated plate for ELISA (Reacti-Bind, Pierce), that had been previously blocked with PBST/10% skim milk for 2 h, and the plate was shaken at room temperature for 1 h. After the liquid was discarded, the plate was washed three times with 200 μl of PBST. One hundred μl of anti-HA antibody diluted with PBST ( 1/2,000) was dispensed, and the plate shaken at room temperature for 1 h. After the liquid was discarded, the plate was washed three times with 200 μl of PBST. One hundred μl of anti-rabbit IgG antibody diluted with PBST ( 1/2000) was dispensed, and the plate was shaken at room temperature for 30 min. After the liquid was discarded, the plate was washed three times with 200 μl of PBST. One hundred μl of the OPD color developing solution was added and the plate was shaken at room temperature for 15 min. Then, 50 μL of 2 N sulfuric acid was added to terminate the reaction, and the absorbances at 492 nm measured using a plate reader (FIG. 21).

Finally, the reaction time in the ELISA-based detection system for the synoviolin autoubiquitination reaction was examined.

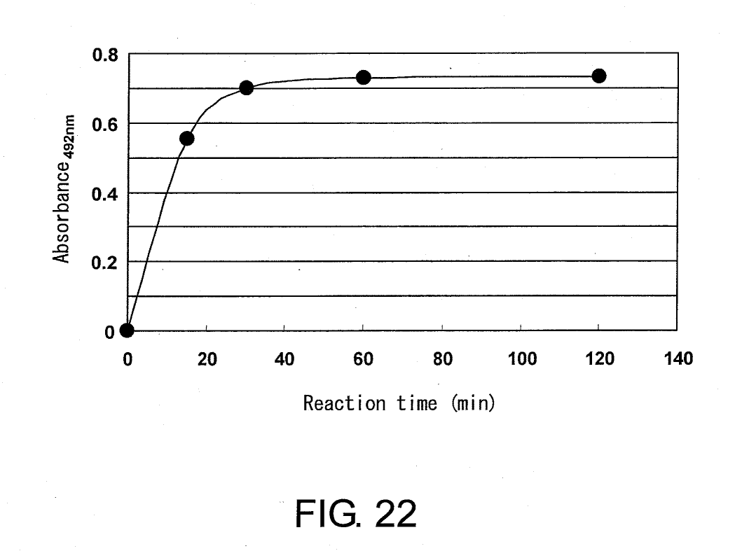

Specifically, ATP (10 mM), His-E1 (human, 125 ng), His-PK-HA-Ub (1.6 μg), His-UBE2G2 (0.8 μg), and GST-Syno ATM (200 ng) were mixed, and the in vitro ubiquitination reaction (30 μL) was performed at 37° C. for 15, 30, 60, and 120 min. After adding 70 L of 0.5M EDTA to the reaction solution to terminate the reaction, the mixed solution was added to a glutathione coated plate for ELISA (Reacti-Bind, Pierce), that had been previously blocked with PBST/10% skim milk for 2 h, and the plate was shaken at room temperature for 1 h. After the liquid was discarded, the plate was washed three times with 200 μl of PBST. One hundred μL of anti-HA antibody diluted with PBST ( 1/1,000) was dispensed to the plate and the plate then shaken at room temperature for 1 h. After the liquid was discarded, the plate was washed three times with 200 μl of PBST. One hundred μL of anti-rabbit IgG antibody diluted with PBST ( 1/2,000) was dispensed to the plate and the plate then shaken at room temperature for 30 min. After the liquid was discarded, the plate was washed three times with 200 μL of PBST. One hundred μL of the OPD color developing solution was added, and the plate was shaken at room temperature for 15 min. Then, 50 μL of 2 N sulfuric acid was added to terminate the reaction, and absorbances at 492 nm were measured using a plate reader (FIG. 22).

The results showed that, in this ELISA system, signals were enhanced in the presence of ATP, depending on the concentration of the anti-HA antibody and the secondary antibody (FIG. 20), the GST-Syno ATM concentration (FIG. 21), and the reaction time (FIG. 22).

### Example 17

### Effects of Low Molecular Weight Compounds on RASCS

In order to evaluate the effects of the two low molecular weight compounds No. 32 (Compound X) and No. 38 (Compound Y), which had been designed by screening using synoviolin autoubiquitination as an index, on RASCs, the effect of these materials on suppression of RASC proliferation was first examined.

The low molecular weight compounds termed No. 32 (Compound X) and No. 38 (Compound Y) were used as specimens. HFLS-RA medium (Cell Applications Inc.; Code CA40405) was used for RASC. RASCs were seeded in a 96-well microtiter plate at 1,000 cells/well, and cultured for 24 h in DMEM containing 10% fetal bovine serum (Kohjin Bio). Then, twofold serial dilutions of the specimens, beginning from a final concentration of 50 μM, were added to the cells. After 1, 24, and 48 h, 3-(4,5-dimethylthiazol-2-yl)-2,5-diphenyltetrazoliumbromide (MTT) was added to a final concentration of 0.5 mg/mL. After 2 h, the medium was aspirated, and the remaining formazan was dissolved with 50 μL of dimethyl sulfoxide. Then, absorbances at 540 μm were measured. The absorbances of the group treated with the specimens were used to calculate cell survival rates, with 0% representing the absorbance of the group without cells or specimen, and 100% representing the absorbance of the group without specimen.

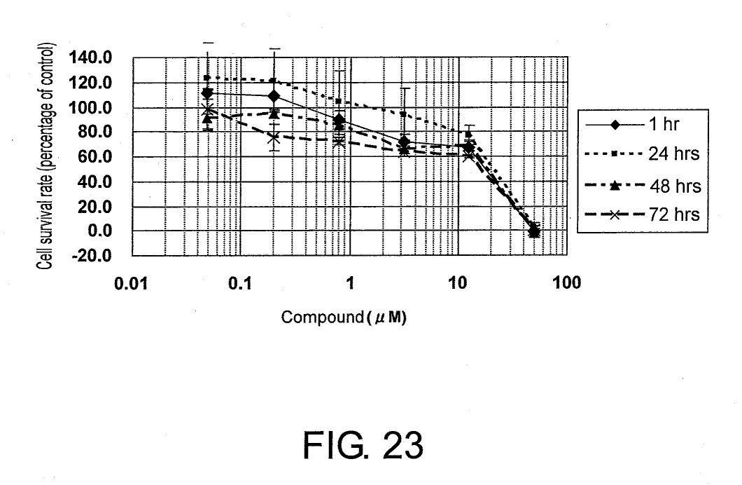

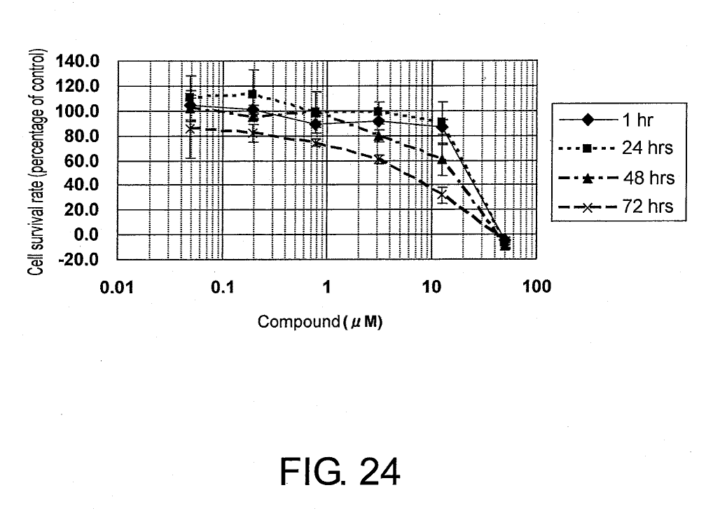

The results showed that the IC₅₀ values of the compounds after 72 h of culture were 20 μM for No. 32 and about 6 μM for No. 38 (FIGS. 23 and 24). These low molecular weight compounds thus suppressed the proliferation of RASCs at low concentrations, and were thus considered to have an anti-rheumatic effect.

### [Sequence Listing Free Text]

SEQ ID NO: 18: synthetic oligonucleotide (DNA/RNA mixture)
SEQ ID NO: 19: synthetic oligonucleotide (DNA/RNA mixture)

### INDUSTRIAL APPLICABILITY

The present invention provides p53 protein-activating agents comprising substances that inhibit the expression or function of a synoviolin protein as active ingredients. These agents are useful as cancer therapeutic agents.

Moreover, the screening methods developed based on the findings discovered by the present inventors can be used to efficiently obtain candidate compounds for cancer therapeutic agents.

Furthermore, various findings discovered by the present inventors are expected to greatly contribute to academic studies seeking to reveal the mechanism of the p53 cancer-suppressing effect.

The p53 protein-activating agents of the present invention are also useful as research reagents for revealing the p53-related cancer-suppressing effect.

Moreover, the present invention provides anti-rheumatic agents comprising substances that regulate the autoubiquitination of synoviolin protein as active ingredients. Also, the screening methods developed based on the findings discovered by the present inventors can be used to efficiently obtain candidate compounds for anti-rheumatic agents.

1. A p53 protein-activating agent, comprising as an active ingredient a substance that inhibits expression and/or function of a synoviolin protein.

2. The agent of claim 1, wherein the substance that inhibits the expression and/or function is a low molecular weight compound having an activity of inhibiting the binding between a synoviolin protein and a p53 protein.

3. The agent of claim 1, wherein the substance that inhibits the expression and/or function is a low molecular weight compound having a function of inhibiting ubiquitination of a p53 protein.

4. The agent of claim 1, wherein the substance that inhibits the expression and/or function is a low molecular weight compound having a function of activating a p53 phosphorylation protein.

5. The agent of claim 4, wherein the p53 phosphorylation protein is ATM or ATR.

6. The agent of claim 1, wherein the substance that inhibits the expression and/or function is a compound selected from the group consisting of:
(a) an antisense nucleic acid against a transcript of a synoviolin gene or a portion of the transcript;
(b) a nucleic acid having a ribozyme activity of specifically cleaving a transcript of a synoviolin gene; and
(c) a nucleic acid having an effect of inhibiting the expression of a synoviolin gene by an RNAi effect.

7. The agent of claim 1, wherein the substance that inhibits the expression and/or function is a compound of (a) or (b):
(a) an antibody which binds to a synoviolin protein and/or a p53 protein; and
(b) a synoviolin protein mutant having a dominant negative property against the synoviolin protein.

8. A cancer therapeutic agent, comprising the agent for activating a p53 protein of any one of claims 1 to 7 as an active ingredient.

9. A method of screening for a cancer therapeutic agent, comprising the steps of:
(a) contacting a test compound with a cell expressing a synoviolin gene;
(b) measuring expression level or activity of a synoviolin protein in the cell; and
(c) selecting a compound that lowers the expression level or activity as compared to when the test compound is not contacted.

10. A method of screening for a cancer therapeutic agent, comprising the steps of:
(a) contacting a test compound with a cell or cell extract which contains DNA having a structure in which a transcriptional regulatory region of a synoviolin gene and a reporter gene are operably linked to each other;
(b) measuring the expression level of the reporter gene; and
(c) selecting a compound that lowers the expression level of the reporter gene as compared to when the test compound is not contacted.

11. A method of screening for a cancer therapeutic agent, comprising the steps of;
(a) contacting a synoviolin protein, a p53 protein, and a test compound;
(b) measuring the binding activity between the synoviolin protein and the p53 protein; and
(c) selecting a compound that lowers the binding activity as compared to when the test compound is not contacted.

12. A method of screening for a cancer therapeutic agent, comprising the steps of:
(a) contacting a synoviolin protein, a p53 protein, and a test compound;
(b) measuring ubiquitination of the p53 protein; and
(c) selecting a compound that lowers the ubiquitination as compared to when the test compound is not contacted.

13. A method of screening for a cancer therapeutic agent, comprising the steps of:
(a) contacting a synoviolin protein, a p53 protein, a p53 phosphorylation protein, and a test compound;
(b) measuring phosphorylation activity of the p53 phosphorylation protein which uses the p53 protein as a substrate; and
(c) selecting a compound that increases the phosphorylation activity as compared to when the test compound is not contacted.

14. The method of claim 13, wherein the p53 phosphorylation protein is ATM or ATR.

15. A kit for screening for a cancer therapeutic agent, comprising as components:
a synoviolin protein; and
a p53 protein.

16. An anti-rheumatic agent comprising as an active ingredient a substance having a function of regulating autoubiquitination of a synoviolin protein.

17. The anti-rheumatic agent of claim 16, wherein the substance having a function of regulating the autoubiquitination of a synoviolin protein is a low molecular weight compound.

18. The anti-rheumatic agent of claim 17, wherein the low molecular weight compound is represented by formula (I) or (II):

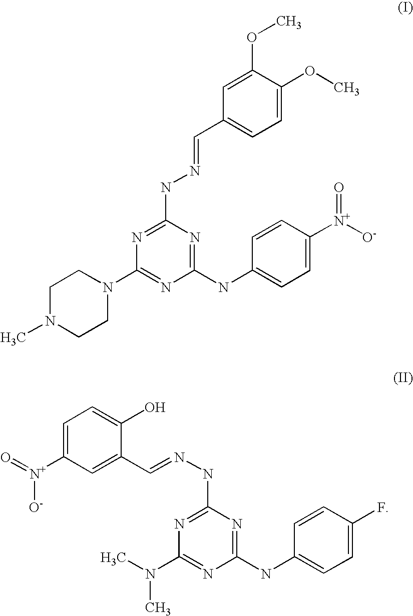

19. The anti-rheumatic agent of claim 18, which has an effect of suppressing proliferation of a rheumatoid arthritis synovial cell.

20. A method of screening for an anti-rheumatic agent, comprising the steps of:
(a) contacting a test compound with a cell expressing a synoviolin gene;
(b) measuring expression level or activity of a synoviolin protein in the cell; and
(c) selecting a compound that lowers the expression level or activity as compared to when the test compound is not contacted.

21. A method of screening for an anti-rheumatic agent, comprising the steps of:
(a) contacting a test compound with a cell or cell extract which contains DNA having a structure in which a transcriptional regulatory region of a synoviolin gene and a reporter gene are operably linked to each other;
(b) measuring the expression level of the reporter gene; and
(c) selecting a compound that lowers the expression level of the reporter gene as compared to when the test compound is not contacted.

22. A method of screening for an anti-rheumatic agent, comprising the steps of:
(a) contacting a synoviolin protein and a test compound;
(b) measuring autoubiquitination of the synoviolin protein; and
(c) selecting a compound regulating the autoubiquitination as compared to when the test compound is not contacted.

23. A kit for screening for an anti-rheumatic agent, comprising a synoviolin protein as a component.
